(19) United States
(12) Patent Application Publication
(10) Pub. No.: US 2014/0127228 A1
(43) Pub. Date: May 8, 2014

(54) INHIBITION OF TGFBETA SIGNALING TO IMPROVE MUSCLE FUNCTION IN CANCER

(71) Applicant: The Trustees Of Columbia University In the City Of New York, (US)

(72) Inventors: Andrew R. MARKS, Larchmont, NY (US); Theresa A. Guise, Indianapolis, IN (US)

(73) Assignees: INDIANA UNIVERSITY SCHOOL OF MEDICINE, Indianapolis, IN (US); THE TRUSTEES OF COLUMBIA UNIVERSITY IN THE CITY OF NEW YORK, New York, NY (US)

(21) Appl. No.: 13/844,073

(22) Filed: Mar. 15, 2013

Related U.S. Application Data
(60) Provisional application No. 61/721,823, filed on Nov. 2, 2012.

(51) Int. Cl. A61K 31/519; A61K 39/395

(52) U.S. Cl. CPC A61K 31/519; A61K 39/3955
USPC 424/158.1; 514/249

(57) ABSTRACT

Methods and compositions useful for the treatment and/or prevention of muscle weakness in cancer patients. In certain embodiments, the methods of the present invention include administering to a cancer patient a therapeutically or prophylactically effective amount of one or more inhibitors of TGFbeta signaling.

### FIELD OF THE INVENTION

The invention relates to methods of preventing and treating muscle weakness in cancer patients, specifically, by administering inhibitors of the TGFbeta signaling pathway.

### BACKGROUND OF THE INVENTION

Muscle weakness and muscle atrophy are common paraneoplastic symptoms in cancer patients. These conditions cause significant fatigue and dramatically reduce patients' quality of life. In addition, they also account for nearly 30% of cancer-related deaths. Muscle weakness is a decrease in the muscle strength, which may be generalized or may affect one muscle or muscle group exclusively. Muscle atrophy (cachexia) is a progressive form of muscle loss. Although both muscle atrophy and muscle weakness cause muscle fatigue, these two pathological conditions are distinct in that the former involves the loss of muscle mass while the latter does not.

One key player in modulating muscle function is ryanodine receptor (RyR). Ryanodine receptors are channels located in the sarcoplasmic reticulum (SR) that open and close to regulate the release of Ca²⁺ from the SR into the intracellular cytoplasm of the cell. The “open probability” (Po) of a RyR receptor refers to the likelihood that the RyR channel is open at any given moment, and therefore capable of releasing Ca²⁺ into the cytoplasm from the SR. There are three types of ryanodine receptors, RyR1, RyR2, and RyR3, among which RyR1 is found predominantly in skeletal muscle as well as other tissues. The RyR channels are formed by four RyR polypeptides in association with four FK506 binding proteins (FKBPs), which stabilize RyR-channel functioning, and facilitate coupled gating between neighboring RyR channels, thereby preventing abnormal activation of the channel during the channel's closed state.

The skeletal muscle ryanodine receptor (RyR1) contains about 30 free thiol residues, rendering it highly sensitive to the cellular redox state. When RyR1 is oxidized such as in aging mice, the RyR1 channel complex becomes “leaky” with increased open probability, leading to intracellular calcium leak in skeletal muscle and causing muscle weakness (Andersson et al., Ryanodine Receptor Oxidation Causes Intracellular Calcium Leak and Muscle Weakness in Aging, Cell Metabolism. Vol. 14/Issue 2, pp. 196-207).

It has been shown that S-nitrosylation of RyR1 and dissociation of the stabilizing subunit calstabin 1 (FKBP12) from RyR1 complex induces SR Ca²⁺ leak and skeletal muscle weakness (Bellinger et al., Hypernitrosylated ryanodine receptor calcium release channels are leaky in dystrophic muscle, Nature Medicine, Vol. 15, Issue 3, pp. 325-330). However, the role of RyR1 channels in muscle weakness in cancer patients is not yet known.

Surprisingly, the inventors found that certain types of cancers, e.g., prostate and breast cancer, create an environment that leads to oxidation of RyR1 which induces it to become “leaky,” suggesting a possible link between leaky RyR1 channels and muscle weakness in cancer patients.

The transforming growth factor-beta (TGFbeta) superfamily consists of a variety of cytokines expressed in many different cell types including skeletal muscle. Members of this superfamily that are of particular importance in skeletal muscle are TGFbeta 1, mitogen-activated protein kinases (MAPKs), and myostatin. These signaling molecules play important roles in skeletal muscle homeostasis and in a variety of inherited and acquired neuromuscular disorders. Although expression of these molecules is linked to normal processes in skeletal muscle such as growth, differentiation, regeneration, and stress response, chronic elevation of TGFbeta1, MAPKs, and myostatin is linked to various features of muscle pathology, including impaired regeneration and atrophy. Mis-regulation of the activity of TGFbeta family members is involved in pathogenesis of cancer, muscular dystrophy, obesity and bone and tooth remodeling. Natural inhibitors for the TGFbeta superfamily regulate fine-tuning of activity of TGFbeta family in vivo. In addition to natural inhibitors for the TGFbeta family, soluble forms of receptors for the TGFbeta family, blocking monoclonal antibodies and small chemical TGFbeta inhibitors have been developed.

Aberrant signaling of TGFbeta1 is found in various skeletal muscle disorders such as Marfan syndrome, muscular dystrophies, sarcopenia, and critical illness myopathy; and inhibition of several members of the TGFbeta signaling pathway has been implicated in ameliorating disease phenotypes, thus suggesting a therapeutic avenues for a large group of neuromuscular disorders (Burks and Cohn. Role of TGF-β Signaling in Inherited and Acquired Myopathies, Skeletal Muscle, 1(1): 19). The dual role of TGFbeta in cancer and muscular disorders has been previously described (Tsuchida et al., Inhibitors of the TGF-beta Superfamily and Their Clinical Applications, Mini Reviews in Medicinal Chemistry, 6(11): 1255-1261, 2006).

Recently, Zhou et al. reported that, in several cancer cachexia models, pharmacological blockade of ActRIIB pathway not only prevents further muscle wasting but also completely reverses prior loss of skeletal muscle and cancer-induced cardiac atrophy (Reversal of Cancer Cachexia and Muscle Wasting by ActRIIB Antagonism Leads to Prolonged Survival, Cell, 142:531-543, 2010). ActRIIB is a high affinity activin type 2 receptor and mediates the signaling by a subset of TGFbeta family ligands including myostatin, activin, GDF11 and others (Lee and McPherron, Regulation of myostatin activity and muscle growth, Proc. Natl. Acad. Sci. USA 98:9306-9311, 2001; Souza et al., Proteomic identification and functional validation of activins and bone morphogenetic protein 11 as candidate novel muscle mass regulators, Mol. Endocrinol., 22:2689-2702, 2008). Zhou et al. showed that, in severely atrophied muscles, some quiescent satellite cells with strong proliferative potential are present and can grow rapidly upon ActRIIB blockade.

Currently, there is no known therapy for treating muscle weakness in cancer patients. There is therefore a need in the art for new and improved methods of preventing and treating muscle weakness in patients with cancer. The present invention addresses these and other needs in the art by providing, inter alia, methods useful for preventing and/or improving muscle strength in patients with cancer. These methods involve modulation of the function of skeletal ryanodine receptors with inhibitors of TGFbeta signaling.

### SUMMARY OF THE INVENTION

The present invention provides a method for treating and preventing muscle weakness, based, in part, on the discovery that, in certain types of cancers, e.g., prostate and breast cancer, RyR1 is oxidized which induces it to become “leaky,” and that inhibiting TGFbeta signaling reduces RyR1 oxidation and leakiness.

In some preferred embodiments, the present invention provides a method of treating or preventing muscle weakness in a subject with cancer in need thereof, which comprises administering to the subject a therapeutically or prophylactically effective amount of an inhibitor of TGFbeta signaling. The inhibitor of TGFbeta signaling may be a TGFbeta antibody. Preferably, the inhibitor or antibody is selected from the group consisting of

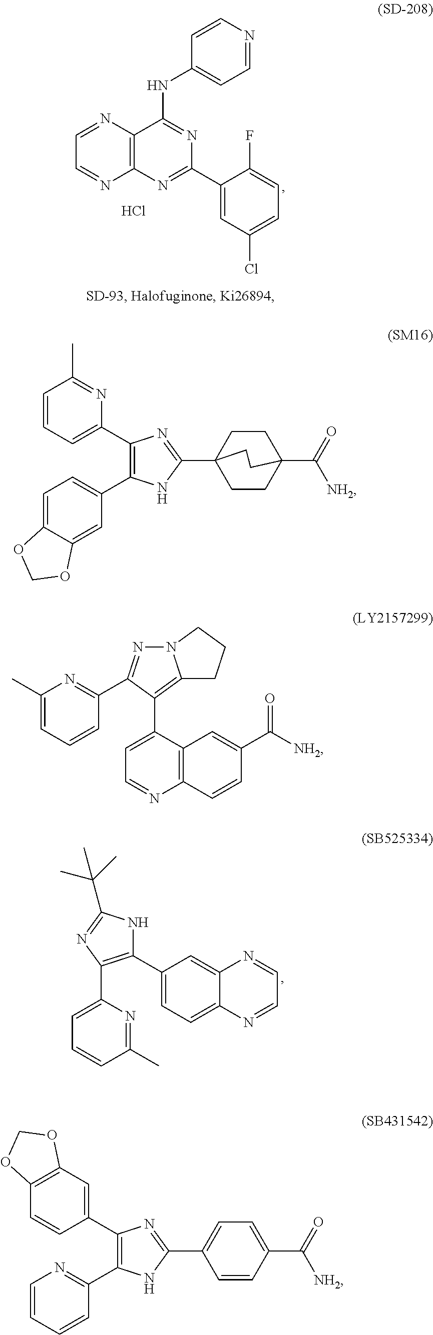 [SMILES: Fc1ccc(Cl)cc1-c1nc(Nc2ccncc2)c2nccnc2n1 ; Cl ; Cc1cccc(-c2nc(C34CCC(C(N)=O)(CC3)CC4)[nH]c2-c2ccc3c(c2)OCO3)n1 ; Cc1cccc(-c2nn3c(c2-c2ccnc4ccc(C(N)=O)cc24)CCC3)n1 ; Cc1cccc(-c2nc(C(C)(C)C)[nH]c2-c2ccc3nccnc3c2)n1 ; NC(=O)c1ccc(-c2nc(-c3ccc4c(c3)OCO4)c(-c3ccccn3)[nH]2)cc1]

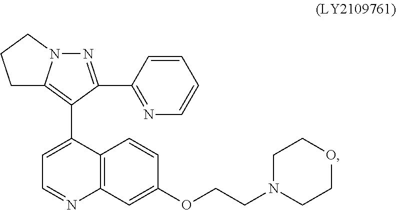 [SMILES: c1ccc(-c2nn3c(c2-c2ccnc4cc(OCCN5CCOCC5)ccc24)CCC3)nc1]

pan-specific TGF-β-neutralizing antibody 1D 11 and a TGFbeta1 antisense oligonucleotide AP 11014.

Most preferably, the inhibitor of TGFbeta signaling is SD-208.

The subject being treated by the method of the invention may suffer from various cancers such as breast cancer, prostate cancer, pancreatic cancer, lung cancer, colon cancer, and gastrointestinal cancer. In some embodiments, the subject has breast cancer.

In other embodiments, the present invention provides a method of treating or preventing muscle weakness in a subject with cancer in need thereof, which comprises administering to the subject a therapeutically or prophylactically effective amount of an inhibitor of TGFbeta signaling that decreases the open probability of the RyR1 channel.

In other embodiments, the present invention provides a method of treating or preventing muscle weakness in a subject with cancer in need thereof, which comprises administering to the subject a therapeutically or prophylactically effective amount of an inhibitor of TGFbeta signaling that decreases Ca²⁺ current through the RyR channel.

In yet another embodiment, the present invention provides a method of treating or preventing muscle weakness in a subject with cancer in need thereof, which comprises administering to the subject a therapeutically or prophylactically effective amount of an inhibitor of TGFbeta signaling that decreases calcium leak through the RyR1 channel.

In a further embodiment, the present invention provides a method of treating or preventing muscle weakness in a subject with cancer in need thereof, which comprises administering to the subject a therapeutically or prophylactically effective amount of an inhibitor of TGFbeta signaling that increases the affinity with which calstabin 1 binds to RyR1.

In an additional embodiment, the present invention provides a method of treating or preventing muscle weakness in a subject with cancer in need thereof, which comprises administering to the subject a therapeutically or prophylactically effective amount of an inhibitor of TGFbeta signaling thereof that decreases dissociation of calstabin 1 from RyR1.

In other embodiments, the present invention provides a method of treating or preventing muscle weakness in a subject with cancer in need thereof, which comprises administering to the subject a therapeutically or prophylactically effective amount of an inhibitor of TGFbeta signaling that increases binding of calstabin 1 to RyR1.

In these methods, the preferred inhibitors of TGFbeta signaling are those that are specifically described and defined by the formulae disclosed herein. The inhibitors can also be administered by way of a pharmaceutical composition or medicament that includes a conventional excipient or carrier.

In certain embodiments, the subject to whom the inhibitors are administered is a mammal selected from the group consisting of primates, rodents, ovine species, bovine species, porcine species, equine species, feline species and canine species. In a preferred embodiment, the subject is a human.

The inhibitors of TGFbeta signaling may be administered by any suitable route known in the art, without limitation. For example, the inhibitors may be administered by a route selected from the group consisting of parenteral, enteral, intravenous, intraarterial, intracardiac, intra intrapericardial, intraosseal, intracutaneous, subcutaneous, intradermal, subdermal, transdermal, intrathecal, intramuscular, intraperitoneal, intrasternal, parenchymatous, oral, sublingual, buccal, rectal, vaginal, inhalational, and intranasal. Additionally, the inhibitors may be administered using a drug-releasing implant.

In one preferred embodiment, the inhibitor of TGFbeta signaling is administered to the subject at a dose sufficient to restore or enhance binding of calstabin 1 to RyR1. For example, when the inhibitor of TGFbeta signaling is SD-208, it may be administered to the subject at a dose of from about 25 mg/kg/day to 100 mg/kg/day, preferably about 40 mg/kg/day to about 80 mg/kg/day, and most preferably 60 mg/kg/day. Other suitable dose ranges are provided in the Detailed Description and Examples. In addition, one of skill in the art can select other suitable doses for administration.

The invention also provides use of an inhibitor of TGFbeta signaling as disclosed herein for preparation of a pharmaceutical composition that includes an excipient or carrier for treating or preventing muscle weakness in a subject with cancer in need thereof.

In other embodiments of the invention, the invention provides use of an inhibitor of TGFbeta signaling for preparation of a medicament for treating or preventing muscle weakness in a subject with cancer in need thereof.

In these methods or uses, the cancer that the subject suffers from is selected from the group consisting of breast, prostate, pancreatic, lung, colon, and gastrointestinal cancers.

In these methods or uses, the inhibitor of TGFbeta signaling is selected from the group consisting of SD-208, SD-93, Halofuginone, Ki26894, SM16, LY2157299, SB525334, SB431542, LY2109761, 1D11 and AP 11014.

The invention also provides the use of an inhibitor of TGFbeta signaling selected from the group consisting of SD-208, SD-93, Halofuginone, Ki26894, SM16, LY2157299, SB525334, SB431542, LY2109761, 1D11 and AP 11014; or a pharmaceutical composition of medicament containing the inhibitor for treating or preventing muscle weakness in a subject with cancer in need thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a diagram showing that prostate cancer metastatic to bone is associated with muscle weakness due to oxidation of RyR1 leading to leaky channels and that sarcoendoplasmic reticulum (SR) calcium transport ATPase (SERCA) and bone-derived TGFbeta plays a key role in this process.

FIG. 2 is a diagram showing the TGFbeta signaling pathway.

FIGS. 3A-H show that decreased muscle specific force production in murine model of human breast cancer metastatic to bone is associated with oxidation of RyR1 calcium channels, dysmorphic mitochondria and SMAD3 activation. A) Nude mice inoculated with MDA-MB-231 human breast cancer cells have lower body weight and osteolytic lesions in the tibia and histology of the femur are shown in X-ray images; B) Tumor bearing mice inoculated with ZR-75-1 human breast cancer cells which cause osteoblastic lesions do not show weight loss; C) Mice with MDA-MB-231 breast cancer bone metastases have decreased muscle specific force in extensor digitorum longus (EDL); D) Tumor-bearing mice with larger osteolytic lesions have weaker muscles and lower body weights; E) Tumor-bearing mice have decreased skeletal muscle cross-sectional area; F) Muscle of mice with MDA-MB-231 osteolytic bone metastases show increased and dysmorphic mitochondria; G) RyR1 is modified and depleted of Calstabin 1 in EDL of Breast Cancer Mice; and H) SMAD3 is phosphorylated in tibialis anterior (TA) muscle lysates from MDA-MB-231 tumor-bearing animals.

FIGS. 4A-K show that decreased muscle specific force production in murine model of human prostate cancer metastatic to bone is associated with oxidation of RyR1 calcium channels and SERCA deactivation. A) Nude mice inoculated with PC-3 human prostate cancer cells show decrease in body weight and osteolytic lesions in the tibia and histology of the femur are shown in X-ray images; B-C) Tumor-bearing mice have decreased skeletal muscle cross-sectional area; D-E) PC-3 tumor bearing mice have decreased body weight D) and decreased grip strength E); F-I) Decreased muscle (EDL) specific force production is improved by SD-208 or ZA. J) RyR1 is oxidized and calstabin 1 is decreased in RyR1 complex from in skeletal muscle (EDL) from mice with metastatic prostate cancer. K) SERCA is oxidized in EDL from prostate cancer mice.

FIGS. 5A-C show LuCAP23.1 prostate cancer model. Panel A shows the body weight change in male mice inoculated intratibially with LuCAP23.1. Panel B compares body weights from normal non-tumor bearing mice, with mice bearing PC-3 bone metastases and LuCAP23.1 bone metastases.

FIGS. 6A-C show that SD-208 prevents PC-3 bone metastases. A) Bone X-rays (4× mag) of both vehicle treated and SD-208 treated mice; B) Osteolytic lesion area of both vehicle treated and SD-208 treated mice; and C) Kaplan-Meier survival curves of both vehicle treated and SD-208 treated mice.

FIG. 7 shows that inhibition of TGFbeta signaling with SD-208 does not increase bone formation and tumor growth in LuCaP 23.1.

### DETAILED DESCRIPTION OF THE INVENTION

The following are definitions of terms used in the present specification.

As used herein and in the appended claims, the singular forms “a,” “an,” and “the” include plural references unless the content clearly dictates otherwise.

The terms an “effective amount,” “sufficient amount,” “therapeutically effective amount,” or “prophylactically effective amount” of an agent or compound, as used herein, refer to amounts sufficient to effect the beneficial or desired results, including clinical results and, as such, the actual “amount” intended will depend upon the context in which it is being applied, such as whether the desired clinical outcome is prevention or treatment. The term “effective amount” also includes that amount of an inhibitor of TGFbeta signaling, which is “therapeutically effective” or “prophylactically effective” and which avoids or substantially attenuates undesirable side effects.

As used herein and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state and remission (whether partial or total), whether detectable or undetectable. “Treatment” can also mean prolonging survival as compared to expected survival if not receiving treatment. Unless otherwise stated, the term “treatment” should be construed as encompassing preventive and therapeutic methods.

The terms “animal,” “subject” and “patient” as used herein include all members of the animal kingdom including, but not limited to, mammals, animals (e.g., cats, dogs, horses, etc.) and humans.

It is to be understood that the inhibitor in this invention may be an antibody.

### Prevention and Treatment of Muscle Weakness in Cancer Patients

As noted herein, currently there is no known cure for muscle weakness in cancer patients, partly due to the lack of understanding of the underlying mechanisms of this pathological condition. Although some of the key players in regulating muscle function are known, their involvement in muscle weakness in cancer patients have not been investigated. One such key player is RyR1 channel protein. The present inventors have shown previously that increased reactive oxygen species (ROS) and/or NO-derived reactive species (RNS) change the redox environment of Ca²⁺ transporters and channels, and thus affect cellular Ca²⁺ cycling.

More recently, the inventors found that RyR1 channels become leaky due to oxidation of the channel in animal models of prostate and breast cancer. More importantly, it was discovered that the oxidation of the RyR1 channel can be inhibited by the inhibitors of the TGFbeta kinase signaling pathway, which significantly improves muscle strength, namely muscle specific force production, in animal models. The inhibition of TGFbeta signaling can be achieved by the use of small molecules such as SD-208, as well as antibodies to TGFbeta. Although these observations were made in breast and prostate cancer models, similar oxidation mechanisms that may affect the same pathways are present in other forms of cancers as well. Noteworthily, the inventors found that muscle weakness in cancer patients correlates directly with metastasis of cancer cells to the bone. For example, cancer patients whose cancer cells have not metastasized to the bone showed no sign of muscle weakness. In contrast, when as few as 10% of the cancer cells were metastasized to the bone, the patients had significant muscle weakness. As TGFbeta signaling is prominent in bone tissues, these observations suggest that TGFbeta signaling plays a major role in causing muscle weakness in cancer patients. Also, to effectively treat muscle weakness in cancer patients, the TGFbeta inhibitor should be administered prior to the time when 25% of the cancer cells were metastasized to the bone, and preferably before 10% of the cancer cells were metastasized to the bone. Of course, the administration of the inhibitor can be administered prior to metastases if possible to prevent later muscle deterioration.

Based on these findings, the present invention provides compositions and methods that are useful for treating and/or preventing muscle weakness and atrophy as well as other muscle related side effects in cancer patients. More particularly, the present invention provides methods of treatment and/or prevention which comprises administration of one or more inhibitors of TGFbeta signaling to cancer patients suffering from, or at risk of developing muscle weakness. In certain embodiments, the methods of the present invention may be used preventively in subjects who are not yet suffering from muscle weakness, but whom exhibit one or more “risk factors” for muscle weakness or are otherwise predisposed to the development of muscle weakness. In addition to cancer, these risk factors can include allergies, anemia, anxiety disorder, asthma, cirrhosis, congestive heart failure, Chronic Obstructive Pulmonary Disease (COPD), depression, diabetes, drug abuse or side effects, HIV infection, kidney failure, malnutrition, obesity, sleep disorder or thyroid disease.

The invention also provides a method for treating other types of muscle disorders and muscular dystrophies (e.g. Emery-Dreifuss, Becker, etc.), by administering one or more inhibitors of TGFbeta signaling. The TGFbeta pathway thus presents a new drug target for molecules and therapies that inhibit this pathway.

Certain inhibitors of the TGFbeta signaling are known in the art. For example, SD-93 and SD-208 are selective chemical inhibitors of the ThRI receptor kinase that inhibit cellular responses to TGFbeta with an IC₅₀ of 20 and 80 nmol/L, respectively (Ge et al., Selective inhibitors of type I receptor kinase block cellular transforming growth factor-h signaling, Biochem Pharmacol, 68:41-50, 2004; Bonniaud et al., Progressive transforming growth factor h1-induced lung fibrosis is blocked by an orally active ALK5 kinase inhibitor, Am J Respir Crit Care Med, 171:889-98, 2005; Uhl et al., SD-208, a novel transforming growth factor h receptor I kinase inhibitor, inhibits growth and invasiveness and enhances immunogenicity of murine and human glioma cells in vitro and in vivo, Cancer Res, 64:7954-61, 2004). In particular, SD-093 and SD-208 belong to a class of highly selective and potent pyridopyrimidine type TβRI kinase inhibitors that block TGF-beta-induced Smad phosphorylation, reporter gene activation, and cellular responses at submicromolar concentrations. These chemicals bind to the ATP-binding site of the TβRI kinase and maintain the enzyme in its inactive configuration (Ge et al., Inhibition of Growth and Metastasis of Mouse Mammary Carcinoma by Selective Inhibitor of Transforming Growth Factor-β Type I Receptor Kinase In vivo, Clin Cancer Res, 12(14): 4315-433, 2006). SD-093 and SD-208 inhibit both tumor growth and metastatic efficiency in vivo of mouse mammary carcinomas.

In a preferred embodiment, the inhibitor of TGFbeta signaling is SD-208.

Other TGFbeta inhibitors known in the art are SD-93, Halofuginone, Ki26894, SM16, LY2157299 (Eli Lilly & Co.); LY2382770 (Eli Lilly & Co.); LY2109761 (Genzyme/Eli Lilly): ANG1122 (Angion Biomedica Corp); AP 11014 (Antisense Pharma GmbH); BTX201 w/ p17 (Biotherapix SLU/Digna Biotech); P144 (Digna Biotech/Flamel/OSDIN); P17 (Digna Biotech/Flamel/OSDIN); SB525334 (GlaxoSmithKline); SB431542 (GlaxoSmithKline/Vanderbilt University); imidazo[2,1-b][1,3,4]thiadiazole derivatives (Merck Co.); and the pan-specific TGF-β-neutralizing antibody 1D11 (Genzyme/Eli Lilly). Structures of some of the inhibitors of TGFbeta signaling are shown below:

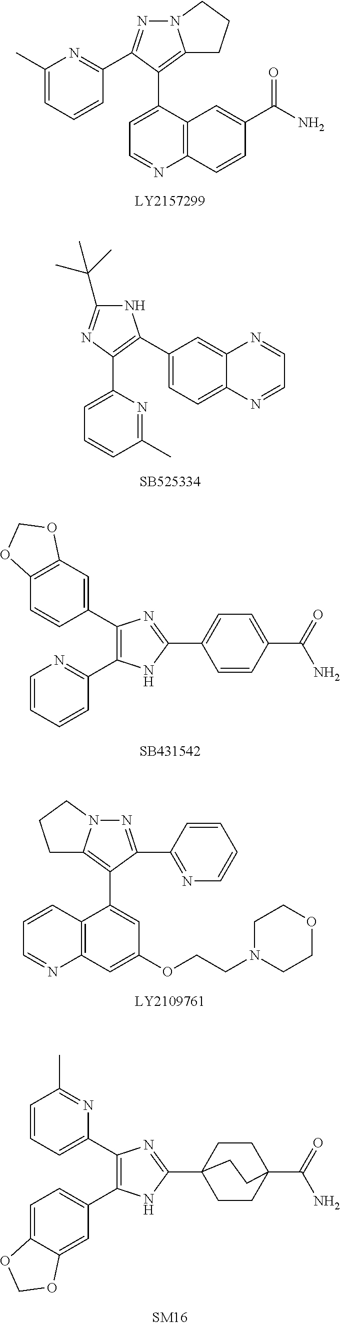 [SMILES: Cc1cccc(-c2nn3c(c2-c2ccnc4ccc(C(N)=O)cc24)CCC3)n1 ; Cc1cccc(-c2nc(C(C)(C)C)[nH]c2-c2ccc3nccnc3c2)n1 ; NC(=O)c1ccc(-c2nc(-c3ccc4c(c3)OCO4)c(-c3ccccn3)[nH]2)cc1 ; c1ccc(-c2nn3c(c2-c2cc(OCCN4CCOCC4)cc4ncccc24)CCC3)nc1 ; Cc1cccc(-c2nc(C34CCC(C(N)=O)(CC3)CC4)[nH]c2-c2ccc3c(c2)OCO3)n1]

AP 11014 is a TGFbeta1 antisense oligonucleotide, which has been shown to significantly reduce TGFbeta1 secretion by 43-100% in different NSCLC (A549, NCI-H661, SW 900), colon cancer (HCT-116) and prostate cancer (DU-145, PC-3) cell lines (Schlingensiepen et al., The TGF-beta1 antisense oligonucleotide AP 11014 for the treatment of non-small cell lung, colorectal and prostate cancer: Preclinical studies, Journal of Clinical Oncology, 2004 ASCO Annual Meeting Proceedings (Post-Meeting Edition), 22(14S): 3132, 2004).

Halofuginone (Hfg) is a synthetic derivative of the plant alkaloid febrifugine, a traditional Chinese herbal medicine. Hfg increases expression of Smad7, an intracellular inhibitor of TGF-beta signaling. In cancer animal models, it shows anti-angiogenic, anti-metastatic and anti-proliferative effects. Hfg has been widely used as a veterinary agent with an excellent safety profile. Recently, the inventors have shown that Hfg therapy decreases development and progression of bone metastasis caused by melanoma cells through inhibition of TGF-β signaling (Juárez et al., Halofuginone inhibits the establishment and progression of melanoma bone metastases, Cancer Res., 72(23):6247-56, 2012).

TGFbeta derived from bone fuels melanoma bone metastasis by inducing tumor secretion of pro-metastatic factors that act on bone cells to change the skeletal microenvironment. The inventors found that Hfg treatment of human melanoma cells inhibited cell proliferation, phosphorylation of SMAD proteins in response to TGFbeta, and TGFbeta-induced SMAD-driven transcription, in addition to reducing expression of TGFbeta target genes that enhance bone metastasis, including PTHrP, CTGF, CXCR4, and IL11. Also, cell apoptosis was found to be increased in response to Hfg. In nude mice inoculated with 1205Lu melanoma cells, a preventive protocol with Hfg were found to inhibited bone metastasis. The beneficial effects of Hfg treatment were comparable to those observed with other anti-TGFbeta strategies, including systemic administration of SD-208, a small molecule inhibitor of TGFbeta receptor I kinase, or forced overexpression of Smad7, a negative regulator of TGFbeta signaling. Significantly, even mice with established bone metastasis that were treated with Hfg had significantly less osteolysis than mice receiving placebo assessed by radiographys. Moreover, Hfg treatment were found to reduce melanoma metastasis to the brain, showing the potential of this novel treatment against cancer metastasis. Hfg is active orally and by intraperitoneal injection, and it has completed Phase I clinical trials in cancer patients. It suppresses the phosphorylation and activation of Smad2 and Smad3 by inducting of Smad7. Overexpression of inhibitory Smad7 has been associated with a reduction on invasive capacity in vitro and anchorage-independent growth, and delays subcutaneous tumor growth in nude mice. The present inventors have shown that Smad7 overexpression decreased melanoma bone metastasis, thus is useful for treating and/or preventing muscle weakness and atrophy as well as other muscle related side effects in cancer patients.

### Pharmaceutical Compositions and Medicaments

The inhibitors of TGFbeta signaling of the invention are formulated into pharmaceutical compositions or medicaments for administration to human subjects in a biologically compatible form suitable for administration in vivo. According to one aspect, the present invention provides a pharmaceutical composition comprising the inhibitors mentioned herein in admixture with a pharmaceutically acceptable excipient or carrier. The pharmaceutically acceptable carrier must be “acceptable” in the sense of being compatible with the other ingredients of the composition and not deleterious to the recipient thereof. The pharmaceutically acceptable carrier employed herein is selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. If necessary, pharmaceutical additives, such as antioxidants, aromatics, colorants, flavor-improving agents, preservatives, and sweeteners, are also added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others.

The pharmaceutical formulations of the present invention are prepared by methods well-known in the pharmaceutical arts. For example, the inhibitors disclosed herein are brought into association with a carrier, excipient and/or diluent, as a suspension or solution. Optionally, one or more accessory ingredients (e.g., buffers, flavoring agents, surface active agents, and the like) also are added. The choice of carrier is determined by the solubility and chemical nature of the compounds, chosen route of administration and standard pharmaceutical practice.

The inhibitors disclosed herein are administered to a subject by contacting target cells in vivo in the subject with the compounds. The compounds are contacted with (e.g., introduced into) cells of the subject using known techniques utilized for the introduction and administration of proteins, nucleic acids and other drugs. Examples of methods for contacting the cells with (i.e., treating the cells with) the compounds of the invention include, without limitation, absorption, electroporation, immersion, injection, introduction, liposome delivery, transfection, transfusion, vectors and other drug-delivery vehicles and methods. When the target cells are localized to a particular portion of a subject, it is desirable to introduce the compounds of the invention directly to the cells, by injection or by some other means. The target cells are contained in tissue of a subject and are detected by standard detection methods readily determined from the known art, examples of which include, without limitation, immunological techniques (e.g., immunohistochemical staining), fluorescence imaging techniques, and microscopic techniques.

Additionally, the inhibitors of the present invention are administered to a human or animal subject by known procedures including, without limitation, oral administration, sublingual or buccal administration, parenteral administration, transdermal administration, via inhalation or intranasally, vaginally, rectally, and intramuscularly. The inhibitors of the invention are administered parenterally, by epifascial, intracapsular, intracranial, intracutaneous, intrathecal, intramuscular, intraorbital, intraperitoneal, intraspinal, intrasternal, intravascular, intravenous, parenchymatous, subcutaneous or sublingual injection, or by way of catheter. In one embodiment, the inhibitor is administered to the subject by way of delivery to the subject's muscles.

For oral administration, a formulation of the inhibitors of the invention may be presented as capsules, tablets, powders, granules, or as a suspension or solution. The formulation has conventional additives, such as lactose, mannitol, cornstarch or potato starch. The formulation also is presented with binders, such as crystalline cellulose, cellulose derivatives, acacia, cornstarch or gelatins. Additionally, the formulation is presented with disintegrators, such as cornstarch, potato starch or sodium carboxymethylcellulose. The formulation also is presented with dibasic calcium phosphate anhydrous or sodium starch glycolate. Finally, the formulation is presented with lubricants, such as talc or magnesium stearate.

For parenteral administration (i.e., administration by injection through a route other than the alimentary canal), the inhibitors of the invention are combined with a sterile aqueous solution that is isotonic with the blood of the subject. Such a formulation is prepared by dissolving a solid active ingredient in water containing physiologically-compatible substances, such as sodium chloride, glycine and the like, and having a buffered pH compatible with physiological conditions, so as to produce an aqueous solution, then rendering said solution sterile. The formulation is presented in unit or multi-dose containers, such as sealed ampoules or vials. The formulation is delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual.

For transdermal administration, the inhibitors of the invention are combined with skin penetration enhancers, such as propylene glycol, polyethylene glycol, isopropanol, ethanol, oleic acid, N-methylpyrrolidone and the like, which increase the permeability of the skin to the inhibitors of the invention and permit the inhibitors to penetrate through the skin and into the bloodstream. The inhibitor/enhancer compositions also may be further combined with a polymeric substance, such as ethylcellulose, hydroxypropyl cellulose, ethylene/vinylacetate, polyvinyl pyrrolidone, and the like, to provide the composition in gel form, which are dissolved in a solvent, such as methylene chloride, evaporated to the desired viscosity and then applied to backing material to provide a patch.

In some embodiments, the composition is in unit dose form such as a tablet, capsule or single-dose vial. Suitable unit doses, i.e., therapeutically effective amounts, can be determined during clinical trials designed appropriately for each of the conditions for which administration of a chosen inhibitor is indicated and will, of course, vary depending on the desired clinical endpoint. The present invention also provides articles of manufacture for treating and preventing the development of muscle weakness in a subject. The articles of manufacture comprise a pharmaceutical composition of one or more of the inhibitors disclosed herein. The articles of manufacture are packaged with indications for various disorders that the pharmaceutical compositions are capable of treating and/or preventing. For example, the articles of manufacture comprise a unit dose of a compound disclosed herein that is capable of treating or preventing the development of muscular weakness, along with an indication that the unit dose is capable of treating or preventing muscle weakness.

In accordance with methods of the present invention, the inhibitors disclosed herein are administered to the subject (or are contacted with cells of the subject) in an amount effective to limit or prevent a decrease in the level of RyR-bound FKBP in the subject, particularly in cells of the subject. This amount is readily determined by the skilled artisan, based upon known procedures, including analysis of titration curves established in vivo and methods and assays disclosed herein. In one embodiment, a suitable amount of the inhibitors of the invention effective to limit or prevent a decrease in the level of RyR-bound FKBP in the subject ranges from about 10 mg/kg/day to about 120 mg/kg/day. In another embodiment, from about 20 mg/kg/day to about 100 mg/kg/day is administered. In another embodiment, from about 40 mg/kg/day to about 80 mg/kg/day is administered. In another, preferred embodiment, about 60 mg/kg/day to about 1 mg/kg/day is administered.

### Examples

Skeletal muscle weakness and cachexia are major contributors to impaired quality of life in patients with prostate cancer, particularly in those with bone metastases. Indeed it is well-established that men undergoing androgen deprivation therapy for prostate cancer have profound muscle weakness. While it has been proposed that altered skeletal muscle physiology (Giordano et al., 2003), and protein breakdown account for some of the muscle weakness associated with cancer, the mechanism(s) causing prostate cancer associated muscle weakness remain largely unknown, and there is no effective therapy to improve muscle function in these patients. Cancer cachexia is the most common paraneoplastic syndrome, characterized by loss of muscle, weakness, impaired functional status and decreased quality of life (Fearon, 2011).

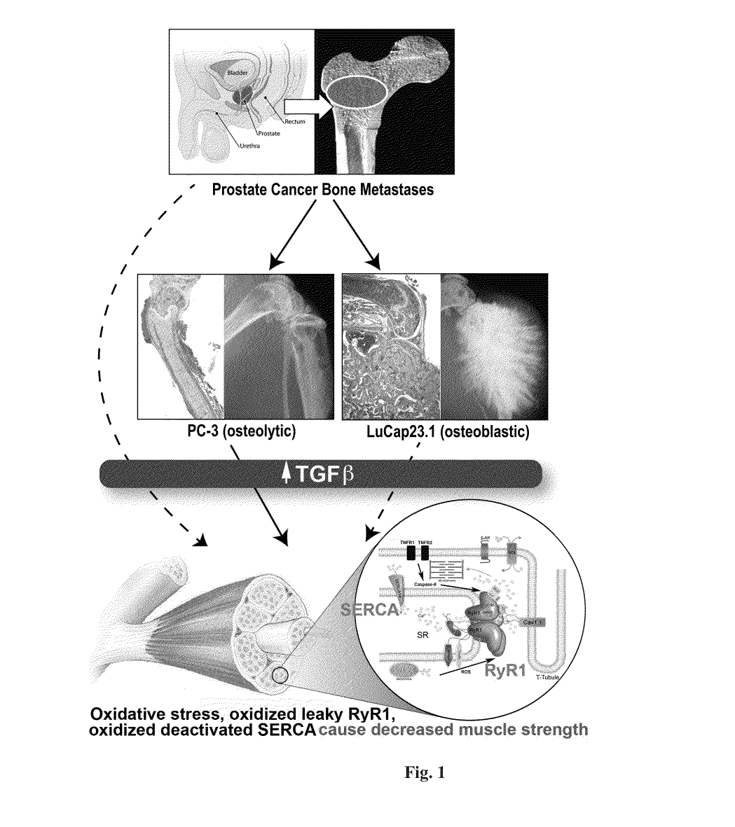

It is hypothesized that prostate cancer-associated muscle dysfunction is due to oxidation-induced molecular changes in muscle that cause impaired muscle contraction due to remodeling of the intracellular calcium release channel/ryanodine receptor (RyR1) on the sarcoplasmic reticulum that is required for skeletal muscle contraction. It has been shown that RyR1 is oxidized and nitrosylated and depleted of the stabilizing subunit calstabin 1 in mice with prostate cancer and prostate cancer metastatic to bone. It is known that the biochemical signature of oxidized/nitrosylated/calstabin 1 depletion results in leaky channels. This defect in RyR1 channel function causes an intracellular Ca²⁺ leak that results in: 1) reduction in the amount of Ca²⁺ stored in the SR which directly reduces the force of skeletal muscle contraction which is dependent on the level of tetanic Ca²⁺ that is released from the SR; 2) mitochondrial Ca²⁺ overload which decreases ATP production and increases reactive oxygen species (ROS) that further oxidize RyR1 and exacerbate the channel leak. Surprisingly, the TGFbeta kinase inhibitor SD-208 (Dunn et al., 2009) administered systemically to mice has been shown to block the prostate cancer induced oxidation/nitrosylation/calstabin 1 depletion of RyR1. It is possible that oxidation by mitochondrial ROS causes RyR1 remodeling manifested by depletion of the stabilizing subunit calstabin 1 from the channel complex resulting in destabilization of the closed state of the channel and intracellular Ca²⁺ leak and deactivation of SERCA (SR Ca²⁺-ATPase). An important aspect of this model is that remodeling of the RyR1 channel is driven by tumor-induced oxidation that is in turn mediated by TGFbeta released from increased bone resorption. This model provides a potential novel therapeutic target for preventing/improving muscle weakness in cancer patients (see FIG. 1).

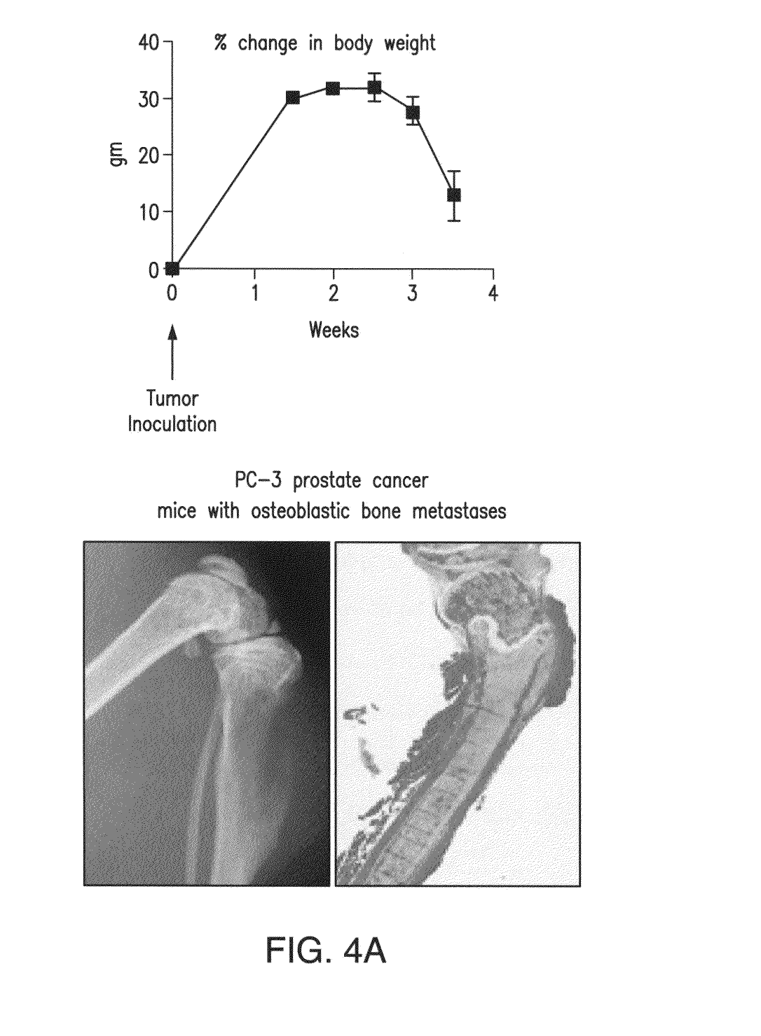

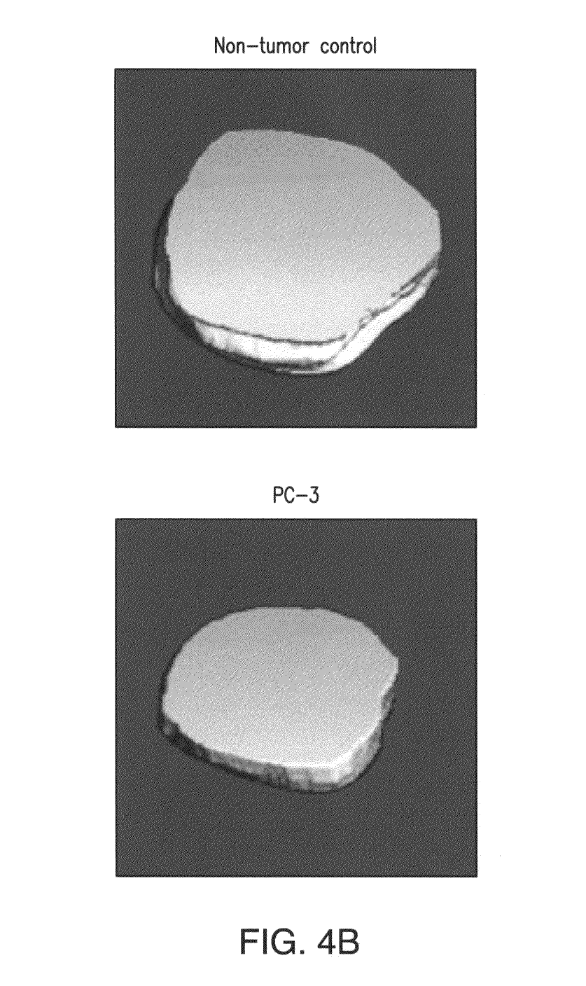

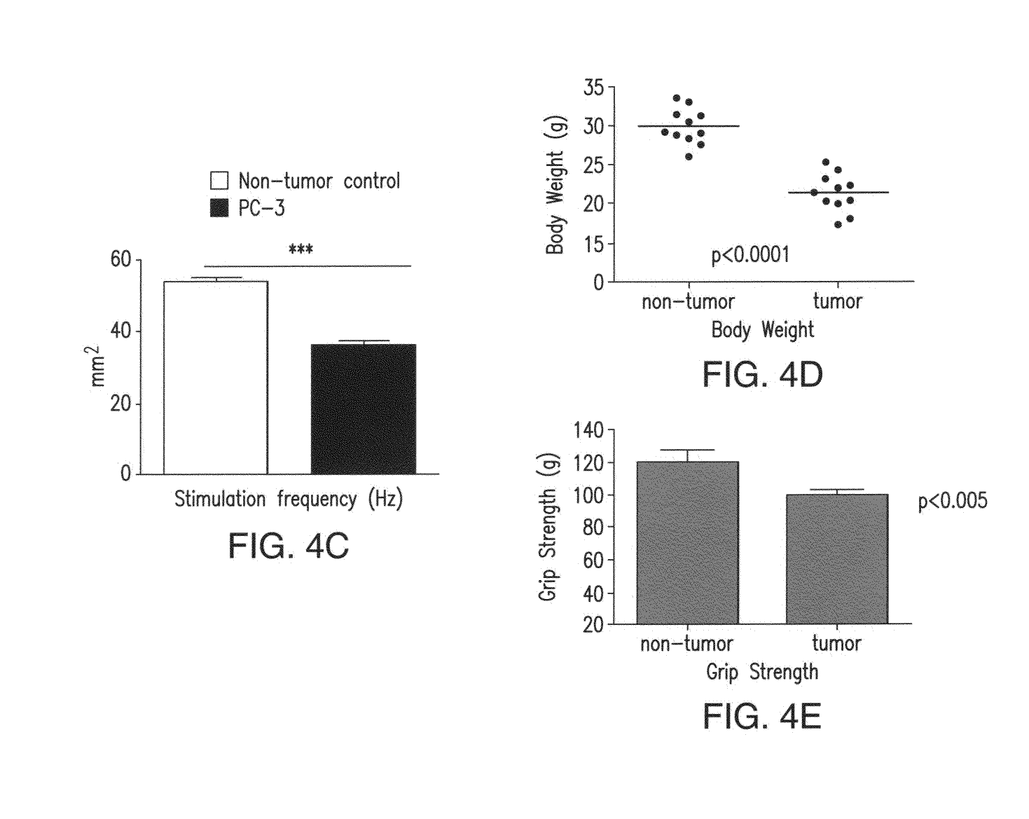

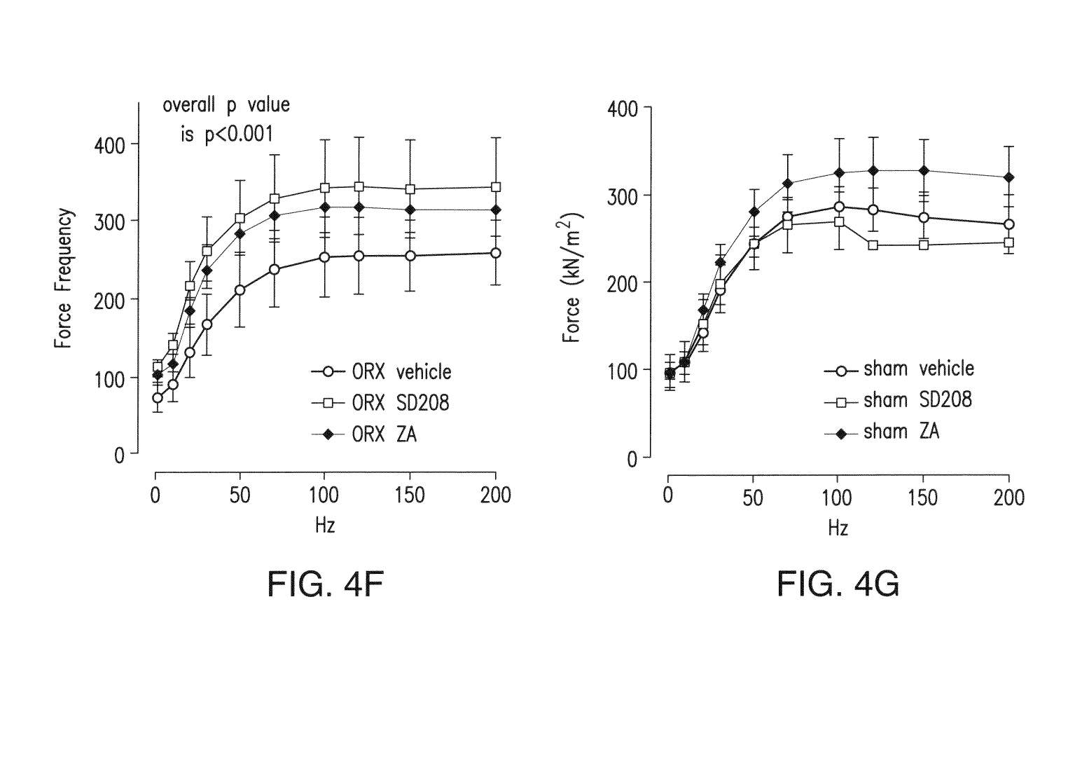

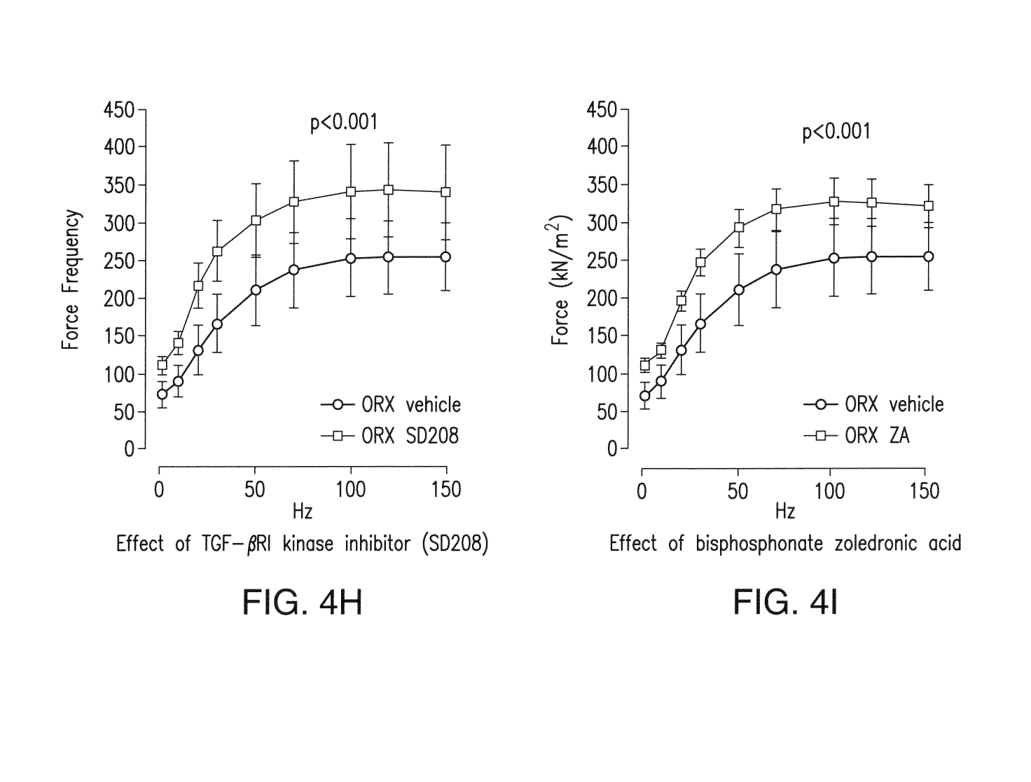

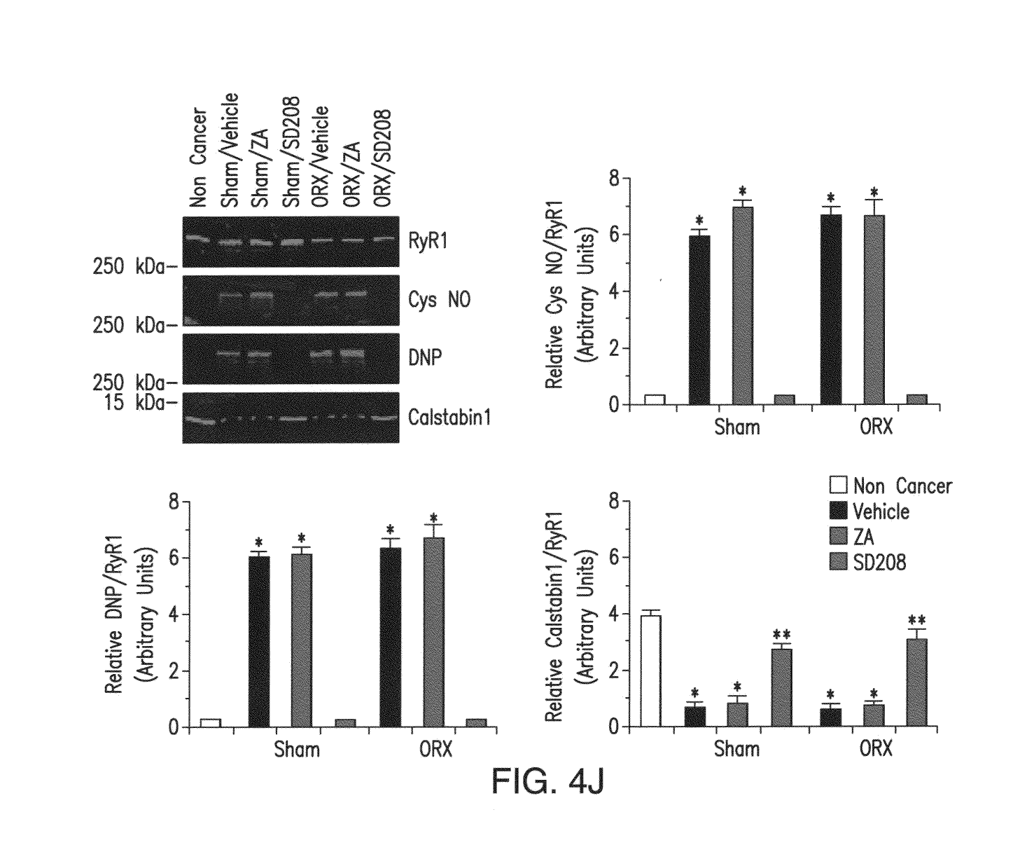

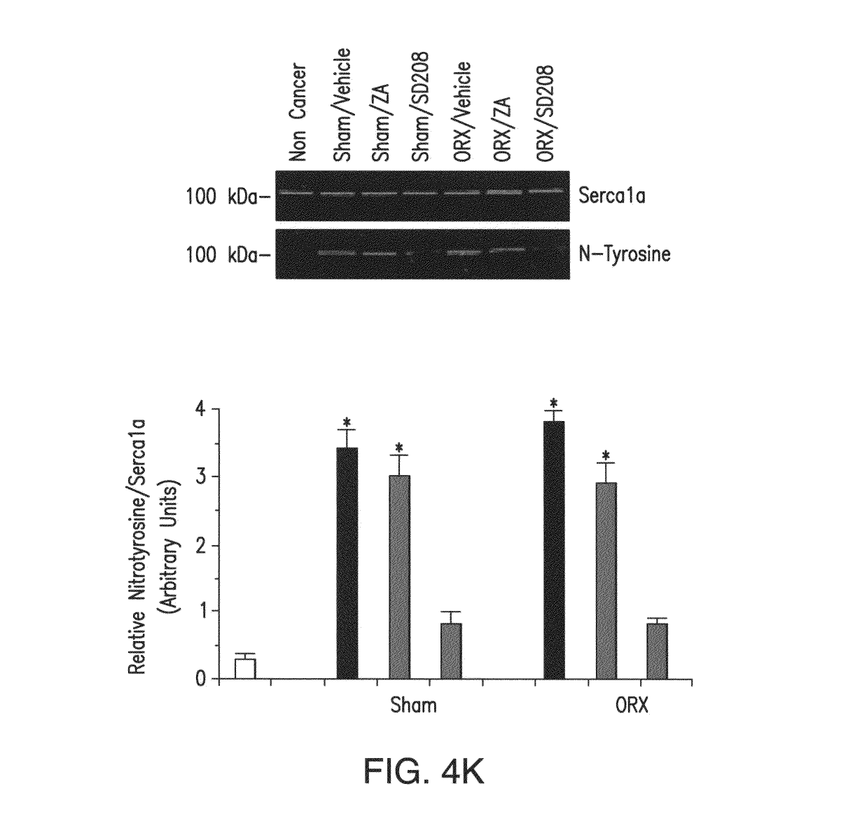

As shown in FIG. 1, prostate cancer metastatic to bone is associated with muscle weakness due to oxidation of RyR1 leading to leaky channels and SERCA, and that bone-derived TGFbeta plays a key role. Oxidation of RyR1 causes SR Ca²⁺ leak which combined with decreased SERCA activity due to oxidation of SERCA depletes SR Ca²⁺, resulting in decreased muscle specific force production, muscle weakness and impaired exercise capacity. Preliminary data show that 1) muscle specific force is reduced in mice with human prostate cancer bone metastases and this is associated with oxidation and nitrosylation of RyR1, depletion of the stabilizing subunit calstabin 1 from RyR1, and hypertrophied and dysmorphic skeletal muscle mitochondria; 2) inhibition of TGFbeta kinase with the small compound SD-208 (Dunn et al., 2009) administered systemically prevents the oxidation/nitrosylation/calstabin 1 depletion of RyR1 (see FIG. 4). The inventors have reported that inflammation and oxidative stress cause remodeling of the skeletal muscle RyR1 channels in muscular dystrophy (Bellinger et al., 2009) and age-dependent sarcopenia (Andersson et al., 2011) resulting in RyR1 channels that leak intracellular Ca²⁺ from the sarcoplasmic reticulum (SR) causing muscle weakness (due to reduced tetanic Ca²⁺), mitochondrial Ca²⁺ overload and mitochondrial dysfunction (including production of reactive oxygen species, ROS), calpain activation and muscle damage.

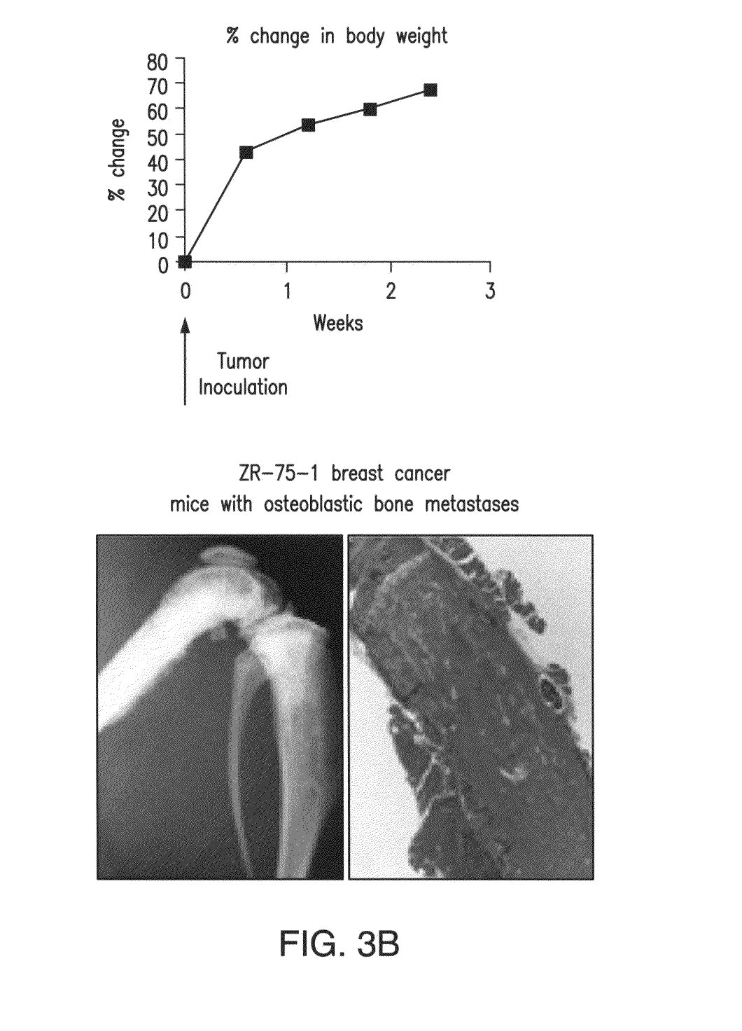

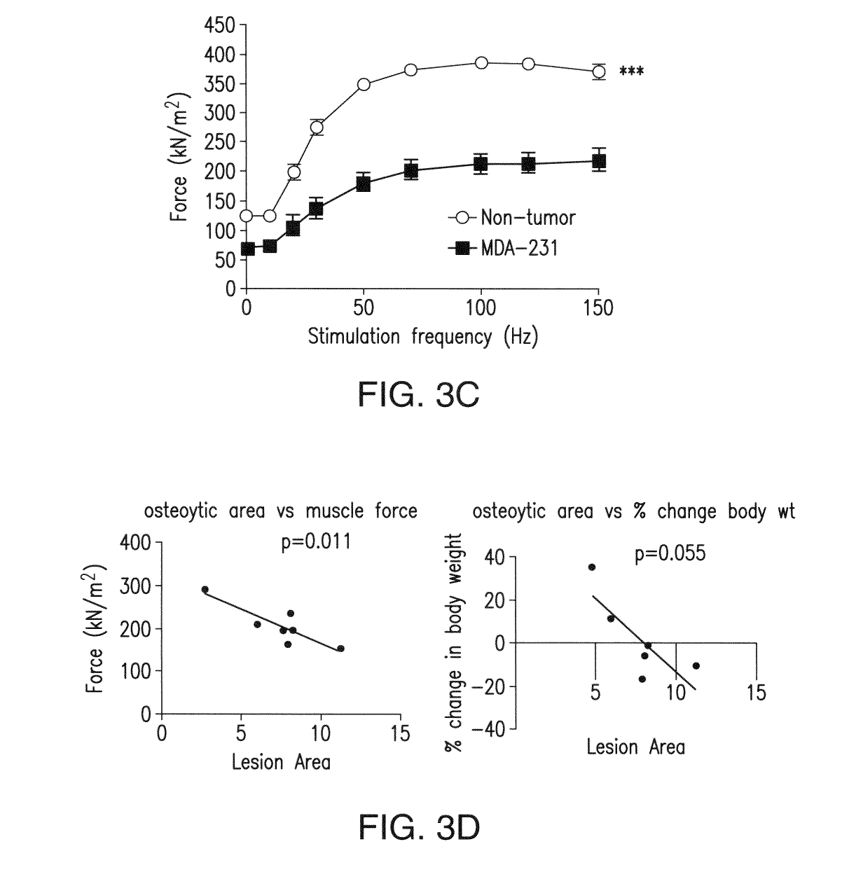

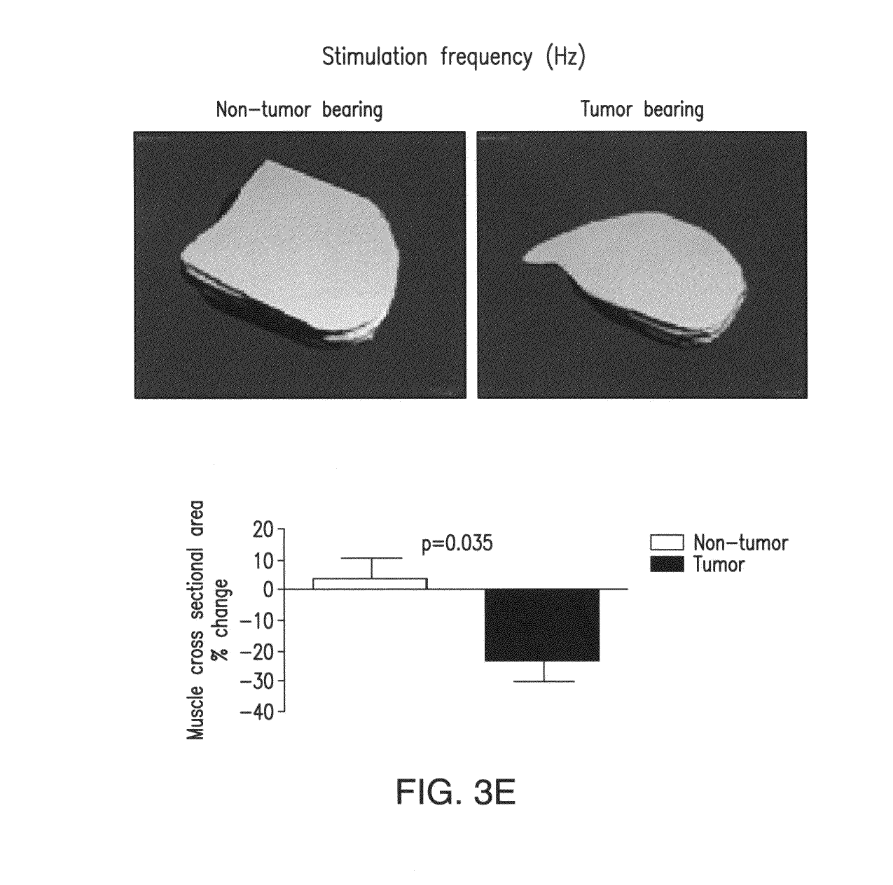

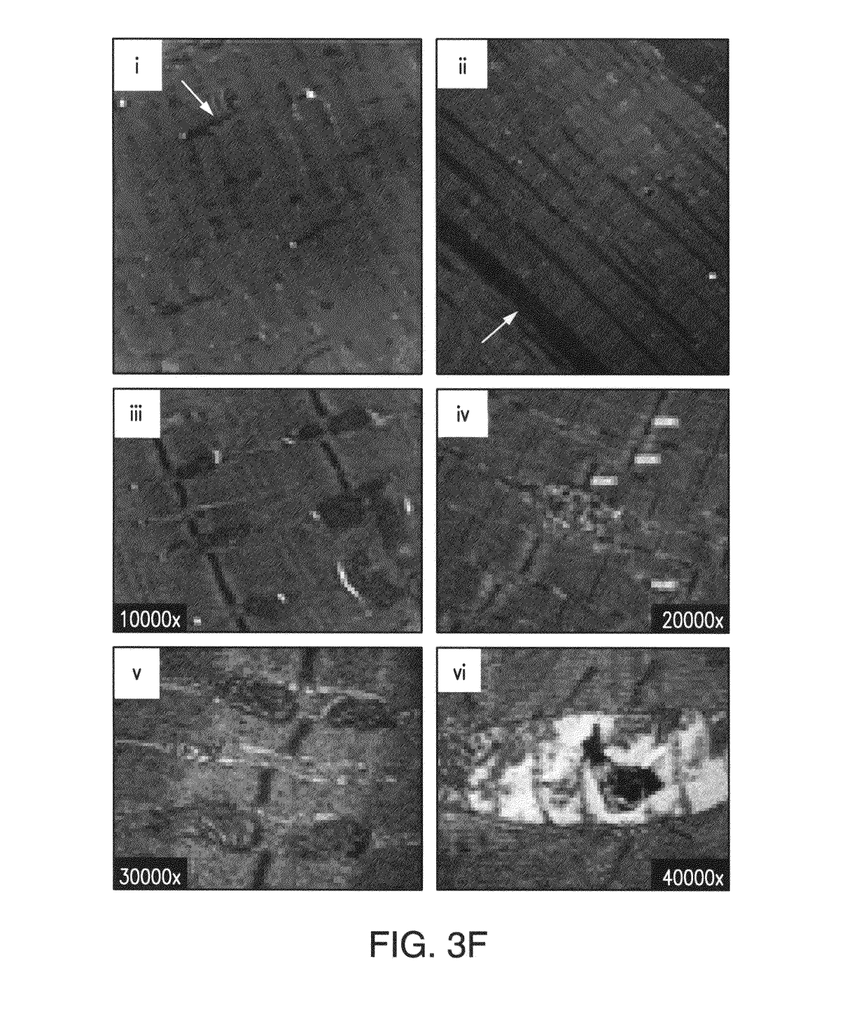

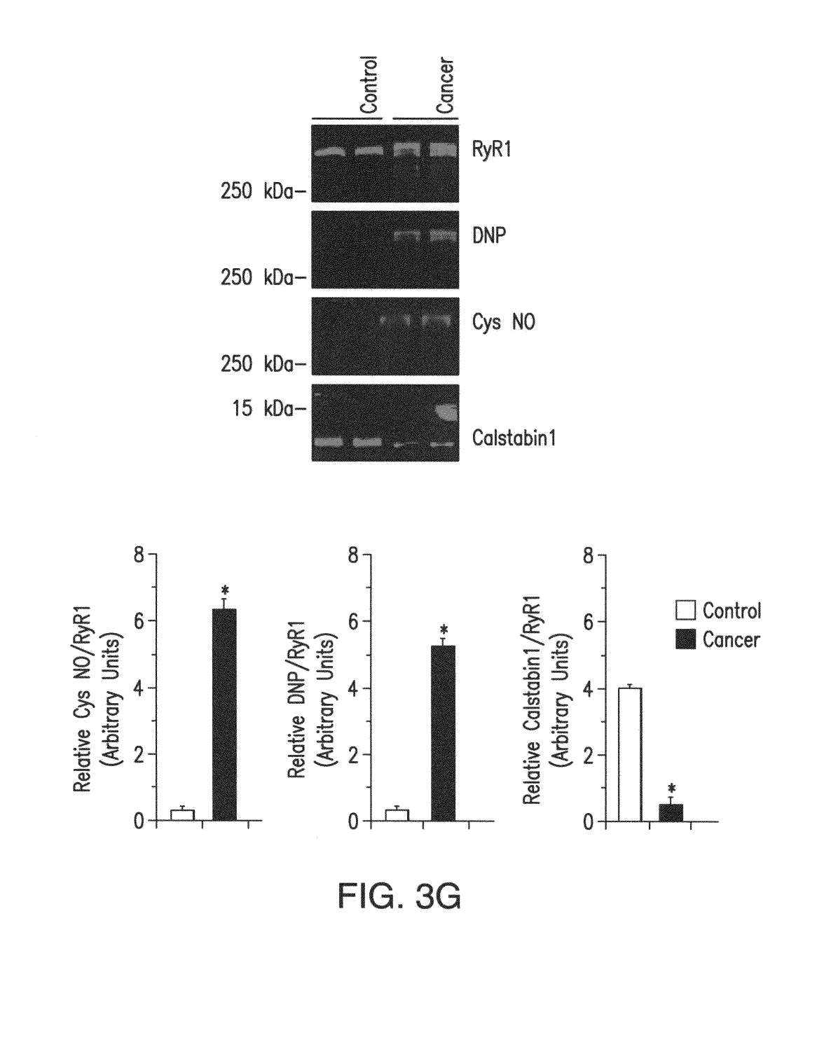

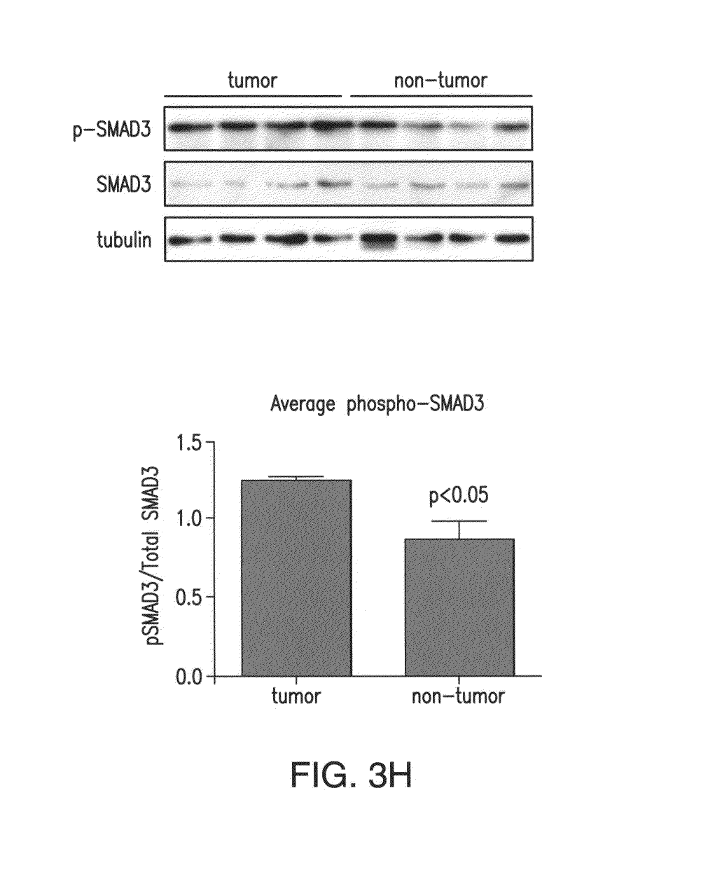

Preliminary data of the inventors (FIG. 4) and that of others (Wang et al., 2003) show that in mice with prostate cancer metastases to bone, cachexia only occurs in mice with osteolytic and not osteoblastic bone metastases. This is also evident in breast cancer bone metastases models, described below (FIG. 3). Thus, cancer-induced oxidation and products of the local tumor-bone microenvironment (specifically due to bone destruction) elaborate factors which may act systemically on muscle to remodel RyR1 channels and induce intracellular SR Ca²⁺ leak. The majority of patients with advanced prostate cancer have bone metastases, and these can be osteolytic, osteoblastic or mixed. Clinical and preclinical data indicate that bone destruction is evident in all patients with bone metastases, regardless of the radiographic appearance (Roudier et al., 2003). Inhibitors of bone resorption (zoledronic acid and denosumab) are effective in preclinical models and reduce skeletal morbidity in patients with prostate cancer bone metastases, regardless of the radiographic phenotype (Corey et al., 2003; Kiefer et al., 2004).

Understanding the mechanisms for prostate cancer-associated muscle dysfunction could result in effective therapy to improve the quality of life in patients with advanced prostate cancer and should have relevance to muscle dysfunction across other tumor types.

### Model of the Effects of Prostate Cancer Induced Oxidative Stress on RyR1 in Skeletal Muscle.

Prostate cancer metastasizes to bone to cause bone destruction (osteolysis; as caused by PC-3) or new bone formation (osteoblastic metastasis; as caused by LuCap23.1). Bone destruction results in the release of factors from bone including TGFbeta.

Prostate cancer is also associated with oxidative stress. All oxidative stress initiates a cascade of events starting with oxidation and nitrosylation of RyR1 and depletion of the calstabin 1 stabilizing protein to induce SR Ca²⁺ leak and reduced muscle force, but this has never been studied in the setting of cancer. In skeletal muscle EC coupling involves the voltage-gated calcium channel, Cav1.1, a voltage sensor that activates RyR1 via direct protein-protein interaction to release SR Ca²⁺ which binds to Troponin C enabling actin-myosin cross-bridging and sarcomere shortening resulting in contraction. Ca²⁺ is pumped back into the SR by SERCA (SERCA1a in fast twitch skeletal muscle and SERCA2a in slow twitch) causing relaxation.

RyR1 is a homotetramer that is regulated by enzymes that are targeted to the channel via anchoring proteins, and other modulatory proteins that are bound to the large cytoplasmic domain (for simplicity these proteins are depicted herein showing only one RyR1 monomer and one of the four Cav1.1 that comprise a tetrad which activates RyR1). The RyR1 complex includes: calstabin 1 (FKBP12) which stabilizes the closed state of the channel (Brillantes et al., 1994), PP1/spinophilin, PKA/PDE4D3/mAKAP, and calmodulin. SR luminal proteins including calsequestrin, triadin and junction also regulate RyR1. RyR1 is activated by stress pathways, including the sympathetic nervous system activation via β-adrenergic receptors (β-AR) resulting in increased Ca²⁺ release and enhanced muscle performance. SR Ca²⁺ release can modulate mitochondrial function, particularly during chronic stress (e.g., muscular dystrophy, aging and cancer), generating ROS that oxidizes RyR1 depleting calstabin 1 from the channel rendering the channels leaky (Kushnir et al., 2010a).

### TGFbeta in Cancer, Bone and Muscle

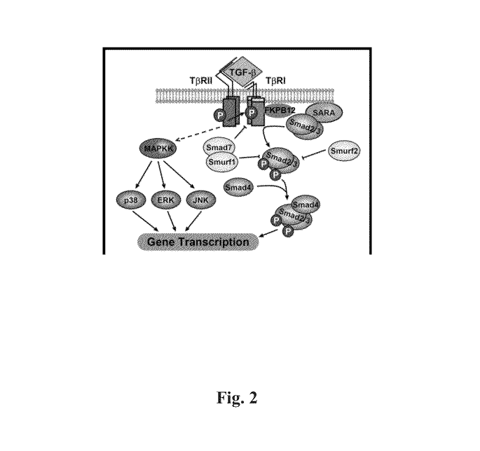

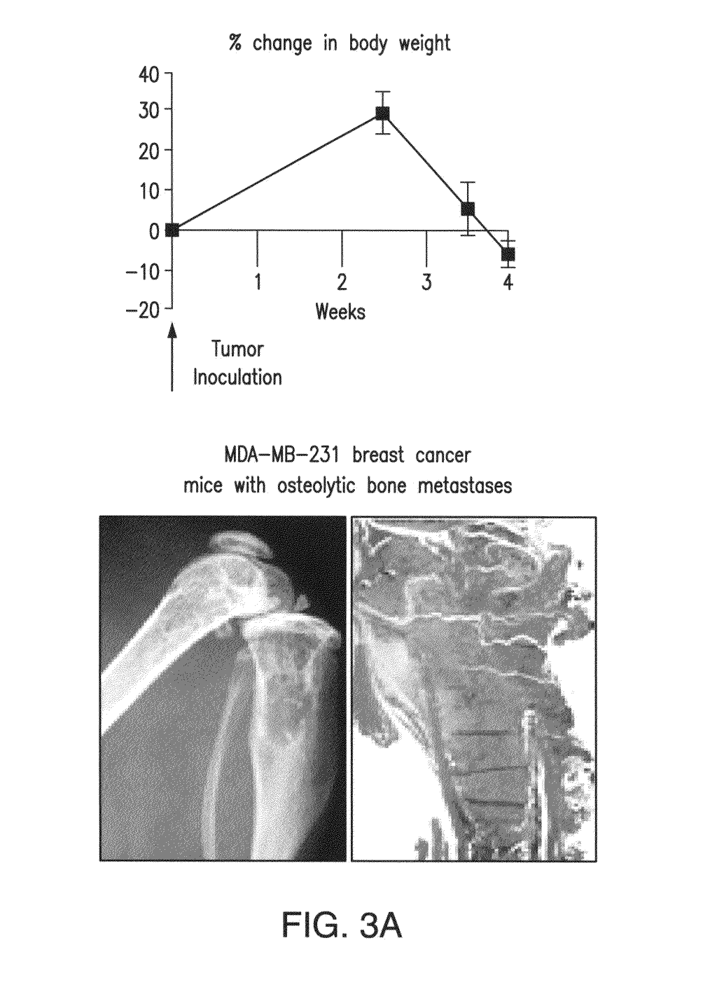

As shown in FIG. 2, TGFbeta promotes bone metastases TGFbeta is a central player in the feedforward cycle of bone metastases. It is synthesized as an inactive precursor immobilized in the bone matrix by osteoblasts and then released and activated by osteoclastic bone resorption (Dallas et al., 2002; Janssens et al., 2005) and is activated by thrombospondin (Murphy-Ullrich and Poczatek, 2000) and proteases (Dallas et al., 2005) from tumors. Tumor TGFbeta signaling increased by the bone microenvironment in vivo (Kang et al., 2005) and breast cancer bone metastases are effectively decreased by TGFbeta signaling blockade (Kakonen et al., 2002; Yin et al., 1999).

TGFbeta Signaling in Cancer and Metastases.

TGFbeta promotes generalized metastases by activating epithelial-mesenchymal transition (Kang and Massague, 2004) and tumor invasion (Desruisseau et al., 1996), increasing angiogenesis (Ananth et al., 1999) and immunosuppression (Thomas and Massague, 2005). TGFbeta mediates effects through binding to the TGFbeta type II receptor (TβRII) and subsequent recruitment of the type I receptor (TβRI) for downstream signaling through multiple parallel pathways (Attisano and Wrana, 2002). No signaling downstream of TGFbeta can occur in the absence of cell surface TβRII expression. Conversely, constitutively active TGFbeta signaling can be mediated by the threonine to aspartic acid mutation in TβRI (T204D). The activated TGFbeta ligand-receptor complex conveys the signal to the nucleus via the phosphorylation and activation of the SMAD signaling pathway (Zhang et al., 1996) as well as other parallel downstream signaling pathways involving RhoA (Bhowmick et al., 2004), stress kinases (i.e. JNK, p38MAPK) (Atfi et al., 1997; Engel et al., 1999; Wang et al., 1997), and others (FIG. 2). The established paradigm of TβRI phosphorylation of Smad2 and Smad3 in conjunction with Smad4 recruitment is important in TGFbeta-mediated G1 cell cycle arrest (Kretzschmar and Massague, 1998). Smad7, also stimulated by TGFbeta, can block Smad2/3 interaction with TβRI and has been shown to promote apoptosis in some cell types. Proposed mechanisms for resistance to TGFbeta include decreased expression of TβRI, TβRII, or TβRIII on melanoma, pancreas, breast, and colon cancers (Buck et al., 2004; Calin et al., 2000; Goggins et al., 1998; Grady et al., 1999; Schmid et al., 1995). Most epithelial-derived tumors become resistant to the growth-inhibitory effect of TGFbeta but retain other aspects of epithelial to mesenchymal differentiation and acquire proliferative response via different mechanisms (Bhowmick et al., 2003; Bhowmick et al., 2001; Blobe et al., 2000; Pasche et al., 1999). Accordingly, TGFbeta antagonists are currently in clinical trials for various cancers, including solid tumor metastases to bone. FKBP12, also known as calstabin, binds to TβRI. Calstabin binds RyR1 to prevent calcium leak. The role of FKBP12 or calstabin in TGFbeta signaling appears to be to prevent ligand independent signaling which is analogous to its prevention of leak through the RyR1 channel.

Although the role of TGFbeta in cancer is complex—tumor suppressor at early stages and metastases promoter in late stages (Elliott and Blobe, 2005), it is an essential mediator of bone metastases (Kang et al., 2003; Muraoka et al., 2002; Yang et al., 2002; Yin et al., 1999). Disruption of the TGFbeta pathway with a dominant negative TβRII or Smad4 knockdown prevented formation of bone metastases in mice (Deckers et al., 2006; Kang et al., 2005; Yin et al., 1999). TGFbeta/Smad signaling is increased in bone metastases of mice as well as in humans. TGFbeta induces tumor secretion of many prometastatic proteins: IL-11, VEGF, ET-1 and PTHrP (Guise and Chirgwin, 2003; Kang et al., 2005; Le Brun et al., 1999; Yin et al., 2003). PTHrP overexpression in breast cancer cells increases bone metastases in mice, and PTHrP antibodies or small molecules that inhibit PTHrP transcription decrease both osteolysis and tumor burden (Gallwitz et al., 2002; Guise et al., 1996a; Kakonen et al., 2002) as do small-molecule inhibitors of TβRI kinase (Bandyopadhyay et al., 2006; Stebbins et al., 2005). Bone-active factors are increased by TGFbeta through both Smad-dependent and -independent pathways (Kakonen et al., 2002).

TGFbeta Effects on Muscle.

Data described herein indicate that TGFbeta signaling is increased in muscle of mice with bone metastases due to PC-3 prostate cancer and MDA-MB-231 breast cancer. Indeed, TGFbeta has been shown to 1) cause muscle atrophy and 2) reduce muscle function and has been implicated in muscle disorders such as muscular dystrophy (Lorts et al., 2012). The mechanism by which TGFbeta mediates these effects at the molecular level is unknown. Halofuginone, an inhibitor of TGFbeta signaling, has been shown to improve muscle function in models of muscular dystrophy (Pines and Halevy, 2011). Other TGFbeta superfamily members have also been implicated in cancer associated muscle dysfunction. For example, myostatin induces skeletal muscle atrophy by up-regulating FoxO1 and Atrogin-1 (but not MuRF 1) through a Smad3-dependent signaling mechanism, myostatin is increased in murine cachexia models (McFarlane et al., 2011), but anti-myostatin therapy has not been shown to improve muscle function in these models (Lokireddy et al., 2012). Further, ActRIIB is a high affinity activin type 2 receptor and mediates the signaling by a subset of TGFbeta family ligands including myostatin and activin. ActRIIB antagonism has been shown to improve muscle mass and survival in mouse models of cachexia (Zhou et al., 2010), but the effect on muscle function was not reported. Such antagonists are currently in clinical trials for muscular dystrophy and other muscle disorder, but have been associated with an increase in blood vessel formation and other significant side effects (reported at ASBMR Bone and Muscle Meeting. Kansas City, July 2012). Thus, improved therapies are necessary to treat muscle dysfunction associated with cancer.

Inventors' data, shown herein, suggest that TGFbeta, released as a consequence of bone destruction in bone metastases, acts systemically to cause muscle dysfunction by increased oxidation of and remodeling of proteins important for excitation-contraction coupling, such as RyR1 and SERCA1a.

### Hypotheses:

The overall hypothesis is that prostate cancer-associated muscle dysfunction is due to oxidation-induced molecular changes in muscle that cause impaired muscle contraction. Three specific hypotheses are tested:

Hypothesis #1: cancer associated skeletal muscle weakness is caused by defective SR calcium release that impairs muscle contraction.
Hypothesis #2: in prostate cancer the oxidative state results in remodeling of EC coupling proteins including the RyR1 channel complex and the SERCA1a Ca²⁺ pump resulting in SR Ca²⁺ depletion and muscle weakness.

Hypothesis #3: skeletal muscle weakness is linked to tumor-induced changes in the bone microenvironment including increased TGFbeta dependent signaling and ROS production that contribute to leaky RyR1 channels and SERCA1a channels with decreased SR Ca²⁺ uptake activity.

There are three specific goals for this experiment:

1) Determine the mechanism of “leaky” ryanodine receptors (RyR1) and deactivated SERCA1a pumps in prostate cancer related loss of skeletal muscle function. RyR1 is an intracellular calcium release channel required for skeletal muscle contraction and SERCA1a pumps Ca²⁺ back into the SR. This goal is achieved by determining whether cancer associated skeletal muscle weakness is caused by a) leaky RyR1 channels due to oxidation of RyR1 by mitochondrial ROS which causes leaky RyR1 due to depletion of the channel of the stabilizing subunit calstabin 1 and b) decreased SERCA1a activity due to oxidation of the SERCA1a pump. In addition, it will be determined whether TGFbeta released from bone or other sources contributes to oxidative stress possibly via activation of nitrogen oxides (NOx). It is shown below that in mice with human breast and prostate cancer bone metastases: 1) extensor digitorum longus (EDL) muscle specific force production is decreased; RyR1 is oxidized, nitrosylated, depleted of calstabin 1, SERCA1a is oxidized (nitrosylated), and skeletal muscle mitochondria are hypertrophied and dysmorphic; 2) inhibition of TGFbeta kinase with the small molecule inhibitor SD-208 prevents oxidation/nitrosylation/calstabin 1 depletion from RyR1 and prevents oxidation of SERCA1a and improves muscle specific force production; and 3) in a mouse model of aging, oxidation of skeletal muscle RyR1 causes depletion of the stabilizing subunit calstabin 1 (FKBP12) from the RyR1 channel complexes and SR Ca²⁺ leak via RyR1 channels (increased open probability and Ca²⁺ spark frequency) and mitochondrial catalase expression blocks RyR1 oxidation indicating that the source of RyR1 oxidation is mitochondrial ROS. Although these data were originally generated in breast cancer models, the inventors now have similar data showing reduced muscle force in mice bearing PC-3 prostate cancer bone metastasis. Preliminary data indicate the muscle force is further reduced if the mice are hypogonadal. This is relevant as most patients with advanced prostate cancer are treated with androgen deprivation therapy.

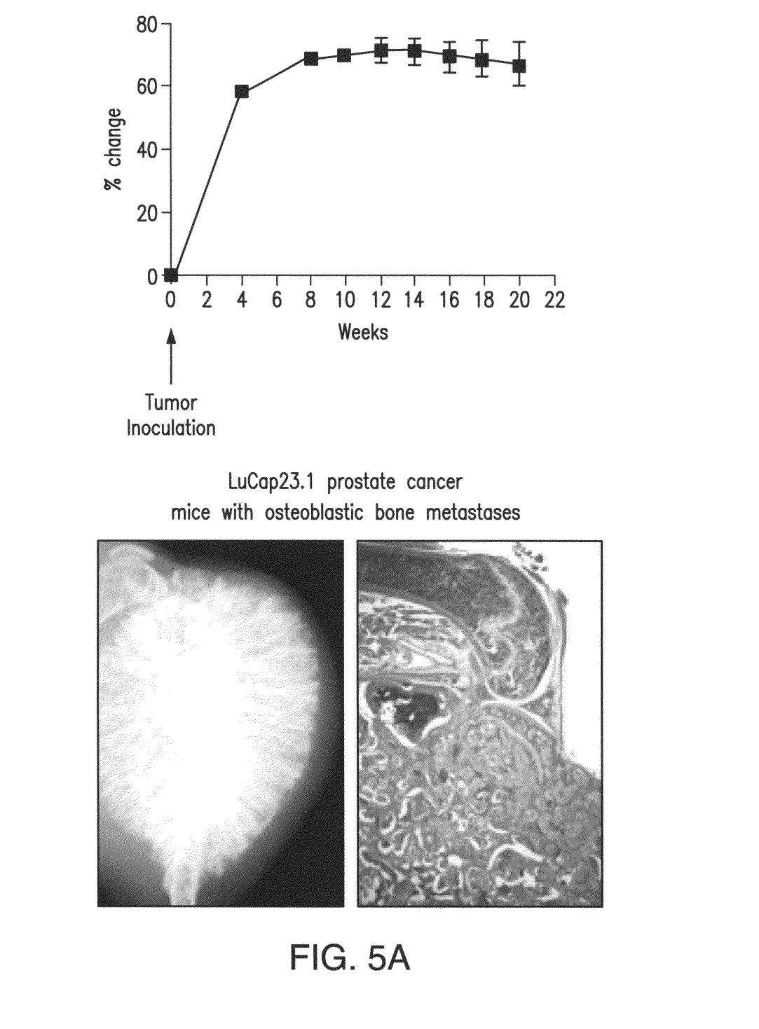

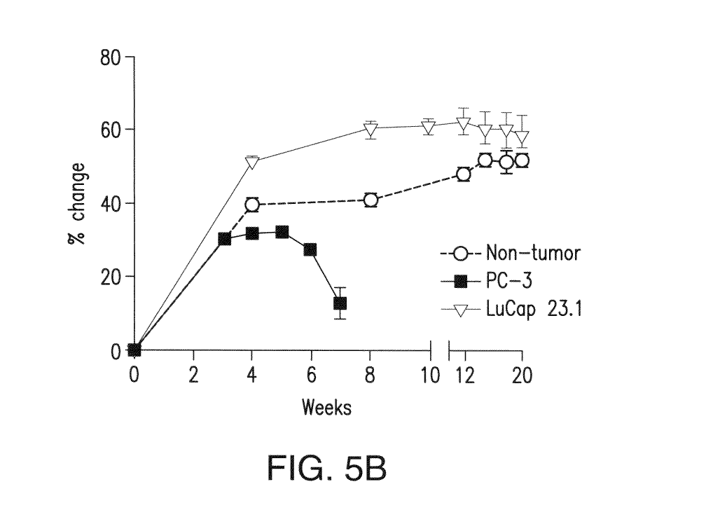

Two human prostate cancer models, PC-3 and LuCAP23.1, which metastasize to bone resulting in osteolytic lesions (PC-3) or osteoblastic lesions (LuCAP23.1) are used to determine the cause of leaky RyR1 channels and decreased SERCA1a activity. Mice are studied in hypogonadal as well as eugonadal states as the former will better represent patients on androgen deprivation therapy. Data shown below indicate that cachexia only occurs in the osteolytic model, PC-3, and not the osteoblastic model, LuCAP23.1 (FIGS. 4 and 5).

Specifically, skeletal muscle specific force production, grip strength and RyR1 channel complexes and function are examined using biochemical and biophysical techniques to determine whether prostate cancer metastatic to bone causes oxidation/nitrosylation/calstabin depletion from RyR1 that results in leaky channels and intracellular (SR) calcium leak that leads to muscle weakness. SERCA pumps from skeletal muscles including but not limited to EDL are examined for oxidation of SERCA1a and SERCA2a and SERCA activity is determined to see whether it is decreased in skeletal muscle from mice with metastatic prostate cancer and whether the oxidation and decreased activity of SERCA can be reversed and/or prevented by treatment with the TGFbeta kinase inhibitor SD-208.

The second goal of this experiment is to determine whether preventing RyR1 channel leak using SD-208, a TGFbeta kinase inhibitor, improves skeletal muscle function in mice with metastatic prostate cancer. The hypothesis is that in prostate cancer an oxidative state results in remodeling of the RyR1 channel complex and intracellular Ca²⁺ leak and oxidation of SERCA1a that causes muscle weakness. It will be determined whether muscle specific force production, grip strength and voluntary exercise are increased in mice with prostate cancer metastatic to bone treated with SD-208.

Inhibition of oxidative signals mediated by TGFbeta could prevent oxidation/nitrosylation/calstabin 1 depletion from RyR1 and could prevent oxidation of SERCA1a which decreases the SR Ca²⁺-ATPase activity. Data presented below show that a) in a mouse model of prostate cancer metastatic to bone SD-208 prevented oxidation/nitrosylation/calstabin 1 depletion from RyR1 and prevented oxidation of SERCA1a and increased muscle specific force production.

TGFbeta kinase inhibitor SD-208 has been tested in vivo in the murine models of prostate cancer to determine whether they increase skeletal muscle specific force production, voluntary exercise and grip strength. SD-208 has been tested in the prostate cancer models PC-3 and LuCAP23.1 to treat prostate cancer bone metastases; and has been shown to be highly effective to prevent bone metastases due to PC-3, but not effective against LuCAP23.1 bone metastases.

The third goal of this experiment is to determine whether the bone microenvironment play a key role in prostate cancer associated muscle weakness. The hypothesis is that skeletal muscle weakness is linked to tumor-induced changes in the bone microenvironment that contribute to leaky RyR1 channels and oxidized SERCA1a which decreases the SR Ca²⁺-ATPase activity. Preliminary data show that 1) cachexia is associated with osteolytic, but not osteoblastic bone metastases; and 2) TGFbeta kinase inhibitor SD-208 prevented oxidation/nitrosylation/calstabin 1 depletion from RyR1 and prevented oxidation of SERCA1a and improved muscle specific force production.

The experiments described herein also investigate whether 1) primary prostate cancer without bone metastases causes skeletal muscle weakness; 2) osteoblastic bone metastases are associated with muscle weakness; 3) muscle weakness occurs in the absence of cachexia; and 4) inhibitors of bone resorption ameliorate skeletal muscle weakness in mice osteolytic prostate cancer bone metastases.

### Murine Model of Human Breast Cancer Metastatic to Bone

As shown in FIG. 3, decreased muscle specific force production in murine model of human breast cancer metastatic to bone is associated with oxidation of RyR1 calcium channels, dysmorphic mitochondria and SMAD3 activation. In embodiment A is shown that body weight is decreased in representative nude mice inoculated with MDA-MB-231 human breast cancer cells and that there are osteolytic lesions in the tibia and histology of the femur, as shown in the X-ray images. In embodiment B is shown lack of weight loss in tumor bearing mice inoculated with ZR-75-1 human breast cancer cells which cause osteoblastic lesions. Mice exhibited normal weight gain despite tumor inoculation and osteoblastic lesions. In embodiment C is shown that representative mice with MDA-MB-231 breast cancer bone metastases have decreased muscle specific force EDL: n=7 per group; p<0.001 ANOVA. In embodiment D is shown that representative tumor-bearing mice with larger osteolytic lesions have weaker muscles and lower body weights. In embodiment E is shown decreased skeletal muscle cross-sectional area in tumor-bearing mice. Micro-CT determination of hind limb skeletal muscle cross-sectional area shows a significant reduction in hind limb cross-sectional area, n=14 for non-tumor and tumor groups. Muscle cross sectional area was assessed in live mice using a microCT scanner (vivio40 CT, ScanCo medical) in the upper shaft region of the tibia (2.5 mm in length starting at 500 um below the growth plate). Scanning parameters of 45kVP, 133 μA and 620 ms integration were used to optimize the contrast between muscle and fat tissue. In embodiment F is shown that increased and dysmorphic mitochondria in muscle of mice with MDA-MB-231 osteolytic bone metastases. (i) Control non-tumor bearing mice white arrow indicates normal mitochondria, 4,800 magnification. (ii) White arrow indicates dramatically increased mitochondria in skeletal muscle of tumor bearing mice. (iii,v) Control (non-tumor bearing) EDL muscles were examined by electron microscopy. (iv,vi) EDL from tumor bearing mice exhibited disorganized mitochondria with absent cristae compared to EDL from non-tumor bearing mice. In embodiment G is shown that RyR1 is modified and depleted of Calstabin 1 in EDL of Breast Cancer Mice. RyR1 was immunoprecipitated from 250 μg of EDL homogenate using an anti-RyR antibody (4 μg 5029 Ab), and the immunoprecipitates were separated on SDS-PAGE gels (6% for RyR, 15% for Calstabin).

All immunoblots were developed and quantified with the Odyssey system (LI-COR, Inc., Lincoln, Nebr.) using IR labeled anti-mouse and anti-Rabbit IgG (1:10,000 dilution) secondary antibodies. Immunoblots developed using anti-RyR (clone 34C, 1:5,000), anti-Cys-NO (1:1000, Sigma, St. Louis, Mo.), anti-DNP (to determine RyR1 oxidation. 1:250), and anti-calstabin (1:2500). The three bar graphs depicting the relative nitrosylation of RyR1. * p<0.01 compared to cancer control; the relative oxidation of RyR1. * p<0.01 compared to control; and the relative amount of calstabin 1 bound to RyR1. * p<0.01 compared to control, respectively. N=6 for both groups. In embodiment H is shown that SMAD3 is phosphorylated in tibialis anterior (TA) muscle lysates from MDA-MB-231 tumor bearing animals. Immunoblot of TA whole cell lysates showed phosphorylation of SMAD3 in muscle from tumor bearing animals. Total SMAD3 levels were detected to determine pSMAD3/SMAD3 ratio. Tubulin was detected as a loading control. Quantitation of pSMAD3/SMAD3 ratio is normalized to tubulin loading. Statistical analysis is performed by unpaired t-test.

Impaired Skeletal Muscle Specific Force in Mice with Osteolytic Breast Cancer Bone Metastases:

Cancer cachexia is the most common paraneoplastic syndrome, characterized by loss of muscle, weakness, impaired functional status and decreased quality of life. Inflammatory cytokine production (IL-1, IL-6, TNFα, & IFN-γ) in response to tumor cells may drive the process but little is known about skeletal muscle function in this setting and there is no effective therapy (Fearon, 2011; Zhou et al., 2010). The inventors have found that murine models of solid tumor metastases to bone are characterized by profound weight loss that is associated with tumor progression. In one such model, MDA-MB-231 human breast cancer bone metastases (Guise et al., 1996b; Yin et al., 1999), the inventors reported a significant decrease in muscle specific force production (Mohammad et al., 2011). Five week old female nude mice inoculated with MDA-MB-231 breast cancer cells via intra-cardiac inoculation developed osteolytic lesions 12 days after inoculation. Tumor bearing mice exhibited significant weight loss 4 weeks after inoculation compared to non-tumor bearing controls (FIG. 3A). This was associated with a significant reduction in total body tissue, lean mass and fat in cancer bearing vs. control mice, assessed by Dual-energy X-ray Absorptiometry (DXA) (P<0.01) while total body % lean mass and % fat were unchanged (not shown). Muscle specific force of the extensor digitorum longus (EDL) muscle, corrected for muscle size, was significantly decreased in cancer bearing mice vs. controls (p<0.001) (FIG. 3C). Tumor-bearing mice also exhibited significantly worse muscle fatigue (not shown). The reduction in muscle specific force correlated with larger osteolytic lesions (p<0.05) (FIG. 3D), and there was a significant decrease in body weight (FIG. 3A). Muscle size, assessed in vivo by microCT, was significantly less in tumor-bearing mice (FIG. 3E) 4 weeks after tumor inoculation, compared with normal mice, which gained muscle mass. Further, muscles from mice with bone metastases showed increased and dysmorphic mitochondria compared with non-tumor bearing mice (FIG. 3F).

The reduction in muscle specific force in these cancer models was comparable to that observed in a murine model of sarcopenia (age-related loss of muscle function) in which the inventors have showed that RyR1 oxidation causes intracellular Ca²⁺ leak and muscle weakness (Andersson et al., 2011). It is hypothesized that muscle weakness in breast cancer could be due to an intracellular SR Ca²⁺ leak in skeletal muscle via RyR1. The inventors analyzed RyR1 complex in skeletal muscle from mice with human MDA-MB-231 breast cancer metastases to bone, in which muscle force was reduced (FIG. 3C). Skeletal muscle (EDL) RyR1 from tumor bearing mice were oxidized, nitrosylated and depleted of calstabin 1 (FIG. 3G). Western blots for phospho-SMAD3 on muscle were performed (FIG. 3C) since TGFbeta is released from the bone microenvironment as a consequence of osteolytic bone destruction in bone metastases and it has been shown to cause muscle atrophy and weakness. Consistently, phospho-SMAD3 was increased in muscle from mice with bone metastases compared to non-tumor-bearing controls.

### Ryanodine Receptor/Calcium Release Channels (RyR1) and Skeletal Muscle Excitation Contraction Coupling:

RyR1 comprises a macromolecular signaling complex that integrates signals from upstream pathways and regulate SR Ca²⁺ release (Marx et al., 2000; Reiken et al., 2003b). The RyR1 macromolecular complex includes: cAMP dependent protein kinase A (PKA); the phosphodiesterase PDE4D3; and the phosphatase PP1. The enzymes in the RyR1 complex are targeted to the cytoplasmic domain of the channel by anchoring proteins, specifically muscle A kinase-anchoring protein (mAKAP) which targets PKA and phosphodiesterase 4D3 (PDE4D3) (Lehnart et al., 2005) and spinophilin which targets PP1 to RyR1. In skeletal muscle at least 4-6 RyR channels cluster into dense arrays (Wang et al., 2001), allowing for the generation of Ca²⁺ “sparks” which are short lived, local release events that can either be spontaneous or triggered by Cav1.1 activation (Cannell et al., 1995; Cheng et al., 1993; Tsugorka et al., 1995).

### Inhibiting TGFbeta Signaling as a Novel Therapeutic Approach to Cancer Associated Decrease in Muscle Function.

To test whether muscle weakness in prostate cancer is a result of an intracellular SR Ca²⁺ leak in skeletal muscle via oxidized leaky RyR1, the RyR1 complex in skeletal muscle from mice with prostate cancer metastatic to bone (PC-3, osteolytic tumors), in which muscle force was reduced was analyzed (FIG. 4).

FIGS. 4A-K show that decreased muscle specific force production in murine model of human prostate cancer metastatic to bone is associated with oxidation of RyR1 calcium channels and SERCA deactivation. In embodiment A is shown that body weight is decreased in nude mice inoculated with PC-3 human prostate cancer cells and that osteolytic lesions are present in the tibia and histology of the femur, as shown in X-ray images. In embodiments B and C are shown decreased skeletal muscle cross-sectional area in tumor-bearing mice determined by Micro-CT as described herein. In embodiments D and E are shown decreased body weight (D) and decreased Grip strength (E) in PC-3 tumor bearing mice. In embodiments F-I are shown decreased muscle (EDL) specific force production is improved by SD-208 or ZA. In embodiment J is shown that RyR1 is oxidized and there is decreased calstabin 1 in the RyR complex from, skeletal muscle (EDL) from mice with metastatic prostate cancer. RyR1 was immunoprecipitated from 250 μg of EDL homogenate using an anti-RyR antibody (4 μg 5029 Ab), and the immunoprecipitates were separated on SDS-PAGE gels (6% for RyR, 15% for Calstabin). All immunoblots were developed and quantified with the Odyssey system (LI-COR, Inc., Lincoln, Nebr.) using IR labeled anti-mouse and anti-Rabbit IgG (1:10,000 dilution) secondary antibodies. Immunoblots developed using anti-RyR (clone 34C, 1:5,000), anti-Cys-NO (1:1000, Sigma, St. Louis. MO), anti-DNP (to determine RyR1 oxidation. 1:250), and anticalstabin (1:2500). ZA—zoledronic acid, SD-208—TGFbeta inhibitor, ORX—orchiectomy. Three bar graphs depicting the relative nitrosylation of RyR1. * p<0.01 compared to non cancer control; the relative oxidation of RyR1. * p<0.01 compared to non cancer control; and the relative amount of calstabin 1 bound to RyR1. * p<0.01 compared to non cancer control, **P<0.01 compared to vehicle treatment, respectively, are shown. In embodiment K is shown that SERCA is oxidized in EDL from prostate cancer mice. SERCA was immunoprecipitated from EDL muscle lysates (0.25 mg) with 4 mg of anti-SERCA antibody (Abcam, ab2861). Immunoprecipitates were size-fractionated using 10% PAGE. Immunoblots were developed for total SERCA (Abcam, 1:2500 dilution) and antinitrotyrosine (Abcam, ab78163, 1:000 dilution) using the Odysey system (Li-Cor). Quantification of immunoblots showing the relative nitrotyrosine/SERCA for each group of EDLs tested. *p<0.01 compared to non-cancer controls as analyzed by ANOVA. N=2 for each group.

As shown in FIGS. 4A-K, muscle size and grip strength were also reduced in murine model of human prostate cancer metastatic to bone. Skeletal muscle (EDL) RyR1 from tumor bearing mice were oxidized, nitrosylated and depleted of calstabin 1 (FIG. 4J). Moreover, strikingly the inventors found that the TGFbeta kinase inhibitor SD-208 prevented oxidation of RyR1 and improved muscle specific force production in mice with prostate cancer metastatic to bone that were also hypogonadal (FIGS. 4F, H). Muscle force was also increased in hypogonadal mice with bone metastases treated with the bisphosphonate zoledronic acid, although zoledronic acid did not prevent the skeletal muscle RyR1 oxidation, nitrosylation and depletion of calstabin 1. This result suggests that in addition to bone-derived TGFbeta, other sources of TGFbeta, may also act on muscle. Zoledronic acid itself, is pro-inflammatory, and may thus be associated itself with oxidation and nitrosylation of RyR1. The fact that SD-208 and zoledronic acid were most effective to improve muscle function in hypogonadal mice with PC-3 prostate cancer bone metastases suggests that the high bone turnover state associated with androgen deprivation therapy may itself induce specific changes in muscle function. Finally, since TGFbeta has been shown to oxidize SERCA, SERCA oxidation was also assessed in muscle samples by immunoprecipitation from EDL muscle lysates. Indeed, SD-208 also inhibited oxidation of SERCA (FIG. 4K). Taken together, these data suggest that TGFbeta promotes oxidation of several proteins associated with sarcoplasmic calcium release to promote muscle weakness.

Oxidation of SERCA2a has been shown to be associated with decreased Ca²⁺ ATPase function (Knyushko et al., 2005). This would decrease SR Ca²⁺ uptake and coupled with leaky RyR1 channels would conspire to deplete SR Ca²⁺, reduce titanic Ca²⁺ that determines the force of muscle contraction.

Of note, SD-208 and zoledronic acid were most effective to improve muscle function in hypogonadal mice with PC-3 prostate cancer. It is well known that androgen deprivation therapy is associated with muscle weakness, but the mechanisms for this are unclear. This could be contributing to the muscle weakness in the mice with prostate bone metastases. As such, all experiments are performed in both hypogonadism as well as eugonadal mice. The specific mechanisms of muscle dysfunction associated with androgen deprivation therapy in the absence of cancer metastases could have adverse effects on muscle function, either through similar or different mechanisms.

FIG. 5 shows LuCAP23.1 prostate cancer model. Shown in embodiment A is the body weight change in male mice inoculated intratibially with LuCAP23.1. The radiographic phenotype is osteoblastic, as is the histologic appearance. Shown in embodiment B is the comparison of body weights from normal non-tumor bearing mice, with mice bearing PC-3 bone metastases and LuCAP23.1 bone metastases. PC-3 was obtained from ATCC and LuCAP xenograft was kindly provided by Dr. Robert Vessella, University of Washington.

### General Methods & Summary of Experimental Approach

### Prostate Cancer Models

### Animals.

These experiments utilize two murine models of prostate cancer bone metastases induced by PC-3 (osteolytic and cachectic; FIGS. 4A-K) or LuCAP23.1 (osteoblastic and not cachectic; FIGS. 5A-B).

For all mouse experiments, N=15 for each group of mice for tumors inoculated in the left cardiac ventricle (PC-3) or intratibially (LuCAP23.1) based on power analysis detailed below. Male nude mice are housed in laminar flow isolated hoods with water supplemented with vitamin K and autoclaved mouse chow provided ad libitum. Cells are introduced into mice at 4 wks of age. Animals are anesthetized with ketamine/xylazine and positioned ventral side up.

Multiple modalities of bone imaging including X-ray and μCT are used to longitudinally monitor the bone lesions and confirmed by histology and quantitative histomorphometry as previously described (Dunn et al., 2009; Guise et al., 1996b; Yin et al., 1999). Muscle mass are followed serially in vivo using the Piximus II system (Wiren et al., 2011) and microCT (Manske et al., 2011).

### Drugs.

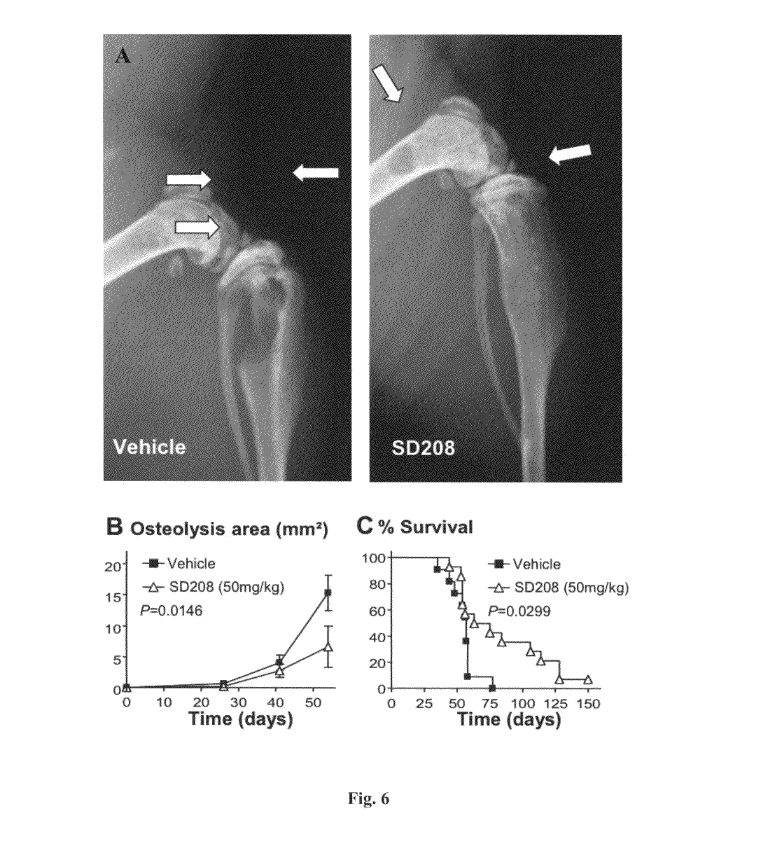

SD-208 is administered by oral gavage (60 mg/kg) as previously described (Dunn et al PLOS ONE). SD208 is used in both PC-3 and LuCAP23.1 to treat bone metastases (see FIGS. 6 & 7).

Statistical methods and data analyses are performed as published previously. Results are expressed as mean±SD. Data for metastasis models are analyzed by repeated measures ANOVA followed by Tukey-Kramer post hoc test using GraphPad Prism. p≦0.05 is significant. Statistical analyses are done in consultation with the IUSCC Biostatistics Core. Details of the methods have been published and demonstrated feasibility (Andersson et al., 2011; Bellinger et al., 2009; Bellinger et al., 2008b; Brillantes et al., 1994; Fauconnier et al., 2011; Kushnir et al., 2010b; Lehnart et al., 2008; Lehnart et al., 2006; Marx et al., 2000; Mohammad et al., 2011; Reiken et al., 2003a; Shan et al., 2010a; Shan et al., 2010b; Wehrens et al., 2003; Wehrens et al., 2005; Wehrens et al., 2006; Wehrens et al., 2004). Assume α error rate=0.05 (probability of type I error) and β error rate=0.20 (probability of type II error), while the mean improvement in specific force production=42% (based on previous studies done by the inventors) and the S.D. of the population studied=1.5, then consider a change in muscle force production of 30% significant. Using the Statmate program, when β=0.20 and α=0.05, the minimum number of animals per group is n=10. Based on the experience of the inventors, approximately 90% of the animals inoculated with tumor cells actually develop tumors; so 15 mice per group are planned, to account for this and unexpected events.

In the experiment, n=15 is used for the intracardiac model. All data groups are analyzed, using established statistical tests, to determine statistical significance of the observations. Student's T-test (paired or unpaired) are used for comparison between two data groups. When more than 2 groups are compared simultaneously analysis of variance (ANOVA), followed by Bonferroni correction, is used (e.g. comparison between control, tumor bearing and tumor bearing+SD-208 groups). In some experiments measurements in a group are repeated over time. For these experiments repeated measurements ANOVA are used to determine statistical significance. All data are expressed as mean±SEM.

### Skeletal Muscle Physiology and Specific Force Production.

Extensor digitorum longus (EDL) muscles are dissected from the hind limbs using micro dissection scissors and forceps. Stainless steel hooks are then tied to the tendons of the muscles using nylon sutures. Thereafter, the muscle is mounted between a force transducer (Harvard apparatus) and an adjustable hook. To quantify the specific force, the absolute force is normalized to the muscle cross-sectional area, calculated as the muscle weight divided by the length and a muscle density constant of 1.056 kg/m as previously described (Yamada et al., 2009).

### Grip Strength.

Forelimb grip strength is accessed after four weeks of treatment as previously described (Bellinger et al., 2009; Bellinger et al., 2008b; Fauconnier et al., 2011). All studies are performed blinded.

### Measurements of Body Composition Using Murine PIXImus II (DXA).

Changes in murine body composition will be measured by densitometry using a mouse DXA scan (PIXImus II, GE Lunar, software version 2.1) as described (Wiren et al., 2011). Quality control calibration using mouse phantom will be performed daily. Under general anesthesia, mice will be placed in prone position on adhesive pad and placed on the PIXImus II for a whole body scan. Lean mass and fat mass will be measured for the whole body and at regions of interest (i.e. leg and back areas). Lean mass and fat mass values will be expressed as a percentage over total tissue mass.

### In Vivo MicroCT to Measure Murine Muscle Mass.

Muscle cross sectional area will be assessed in live mice using a microCT scanner (vivo40 CT, ScanCo medical) as previously described (Manske et al., 2011) and the effects of SD-208 treatment on muscle mass will be assessed non-invasively. Muscle cross sectional area will be assessed in the diaphysial region of the tibia. An area of about 3 mm in length starting at about 2 mm below the growth plate will be scanned. Scanning parameters of 45kVP, 133 μA and 620 ms integration time will be used as a standard setting to optimize the contrast between muscle and fat tissue. Mice are scanned at baseline and at end point and the results will be expressed as percentage change from baseline. Feasibility of this approach has been demonstrated in FIGS. 3 and 4.

### Measure RyR1 Oxidation, Nitrosylation, and PKA Phosphorylation in Skeletal Muscles.

Quantification of RyR1 skeletal channel nitrosylation, oxidation and phosphorylation by protein kinase A (PKA) are performed as described (Bellinger et al., 2009; Bellinger et al., 2008b).

Cellular Oxidation and Mitochondrial Function and Apoptosis in Muscle from Prostate Cancer Mice.

Mitochondrial superoxide production is measured as described (van der Poel et al., 2007) and by using the cell permeable fluorescent indicator MitoSOX Red as previously described (Aydin et al., 2009). Apoptosis will be assessed in skeletal muscles of tumor bearing mice as previously described (Shan et al., 2010c).

### Mitochondrial Assessment in Skeletal Muscle by Electron Microscopy

Skeletal muscle from mice and humans is analyzed via electron microscopy (as in FIG. 3) in the Electron Microscopy core facility, Indiana University, Department of Cell Biology and Anatomy.

Calstabin 1 Depletion from the RyR1 Complex

Quantification of depletion of the channel stabilizing subunit calstabin 1 (FKBP12) from skeletal RyR1 by immunoprecipitation techniques are performed as described (Marx et al., 2000). RyR functional defects (development of “leaky” RyR1 channels). Planar lipid bilayer measurements of prostate cancer cachexia-related changes in skeletal RyR1 single-channel properties are assessed as previously described (Bellinger et al., 2009). Specifically, channel open probability at low activating cis [Ca²⁺]=150 nM are assessed to determine whether RyR1 from tumor bearing mice are “leaky”.

### Ca²⁺-Dependent ATPase Activity.

Ca²⁺-dependent ATPase activity is measured by colorimetric determination of inorganic phosphate as described (Knyushko et al., 2005).

### Test Cancer Associated RyR1 Oxidation in Mcat Mice

Mcat mice are mice with catalase targeted to mitochondria which prevents mitochondrial ROS production. The inventors have previously shown that RyR1 oxidation is blocked in Mcat mouse skeletal muscle (Andersson et al., 2011). The Mcat mice are crossed into Rag1−/− immunodeficient mice for the human prostate tumors to survive and for determining whether prostate cancer associated RyR1 oxidation and muscle weakness are ameliorated in Mcat mice.

### Determining the Mechanism of “Leaky” Ryanodine Receptors (RyR1) in Prostate Cancer Related Loss of Skeletal Muscle Function

It is hypothesized that cancer associated skeletal muscle weakness is caused by leaky RyR1 channels due to oxidation of RyR1 by mitochondrial ROS which depletes the channel of the stabilizing subunit calstabin, and oxidation of SERCA pumps in the SR which decreases SR Ca²⁺ uptake. Both leaky RyR1 channels and deactivated SERCA pumps contribute to depletion of SR Ca²⁺ resulting in decreased tetanic Ca²⁺, reduced muscle force production and impaired exercise capacity in animal models of prostate cancer.

As shown herein and previously, EDL muscle specific force production in mice with human prostate and breast cancer bone metastases is decreased and associated with reduced muscle size, oxidation and nitrosylation of RyR1, depletion of calstabin 1 and hypertrophied and dysmorphic skeletal muscle mitochondria. Also, in a mouse model of aging, oxidation of skeletal muscle RyR1 causes depletion of the stabilizing subunit calstabin 1 (FKBP12) from the RyR1 channel complexes resulting in an SR Ca²⁺ leak via RyR1 channels (increased open probability and Ca²⁺ spark frequency). As mitochondrial catalase expression blocks RyR1 oxidation, the source of oxidation of RyR1 is likely to be mitochondrial ROS.

Two models of prostate cancer are used to determine the cause of leaky RyR1 channels. Mice are inoculated with prostate cancer cells which metastasize to bone and compared with age-matched non-tumor bearing mice. The development of bone metastases are determined by serial radiographs, and muscle mass is assessed in vivo by microCT and body composition by DXA (PIXImus). Serum are collected for cytokine measurement by multiplex analysis (Fauconnier et al., 2011) to determine whether cytokines are increased and associated with muscle dysfunction. The following assays are also performed:

1.1) Muscle force measurement, and grip strength assessment are determined for each group.

1.2) RyR1 channel complexes are assessed specifically to determine whether the RyR1 channels have the “leaky” channel biochemical profile characterized by remodeling of the RyR1 complex such that the channels are oxidized, nitrosylated, and depleted of the stabilizing subunit calstabin 1, using methods previously described (Andersson et al., 2011). Muscle mitochondria are analyzed by electron microscopy, as in FIG. 3.

1.3) RyR1 single channel function in planar lipid bilayers are assessed using methods well established in the inventors' laboratory (Andersson et al., 2011). Specifically, the open probability of the RyR1 channels from prostate cancer mice is analyzed to see if it is increased under baseline (resting) conditions which is indicative of channel leak. Ca²⁺ imaging (Ca²⁺ spark measurements) is used to assess the leak of the RyR1 channels as previously described (Andersson et al., 2011). The SR Ca²⁺ content is measured to determine whether the leaky RyR channels cause SR Ca²⁺ depletion and tetanic Ca²⁺ measurements are made to monitor the leaky RyR1, SR Ca²⁺ depletion and tetanic Ca²⁺ reduction, which would reduce muscle force production.

1.4) SERCA 1a and SERCA2a oxidation and function are also assessed.

Determine Whether Preventing RyR1 Channel Leak Using TGFbeta Inhibitors Improves Skeletal Muscle Function in Mice with Prostate Cancer Bone Metastases

The test is conducted to determine whether targeting the molecular mechanism underlying skeletal muscle RyR channel leak in prostate cancer will improve muscle function. It is hypothesized that, in prostate cancer, an oxidative state results in remodeling of the RyR1 channel complex and intracellular Ca²⁺ leak, which causes muscle weakness. Specifically, a novel TGFbeta kinase inhibitor SD-208 is used to determine whether inhibiting TGFbeta dependent signaling can prevent oxidation of RyR1 and SERCA in skeletal muscles from tumor bearing mice and whether this is associated with increased muscle specific force production and grip strength in these two prostate cancer models.

As shown in FIGS. 6 and 7, respectively, TGFbeta inhibitor SD-208 can improve prostate cancer related skeletal muscle weakness. In particular, SD-208 prevents PC-3 bone metastases, as shown by the bone X-rays (4× mag) of both vehicle treated and SD-208 treated mice (FIG. 6A); osteolytic lesion area, Ave±SE, by 2-way ANOVA (FIG. 6B); and Kaplan-Meier survival curves (FIG. 6C). In this experiment, mice were inoculated with 105 PC3 cells and given 50 mg/kg SD-208 (n=14) or vehicle (n=11) 2 days prior to inoculation.

FIG. 7 shows that TGFbeta inhibition with SD-208 does not increase bone formation or tumor growth in LuCaP 23.1. Four-week old, male nude mice were inoculated into the tibia with LuCaP 23.1 human prostate cancer osteoblastic xenograft (n=14-15/group). Mice received SD-208 (50 or 150 mg/kg/day, po) at 12 weeks post tumor inoculation when tumors were evident and continued for 26 weeks throughout the protocol.

### Determining Whether the Bone Microenvironment Plays a Key Role in Causing Skeletal Muscle Weakness in Prostate Cancer Models.

Skeletal muscle weakness is linked to tumor induced changes in the bone microenvironment that contribute to leaky RyR1 channels. It has been shown that cachexia is associated with osteolytic, but not osteoblastic bone metastases. Bone destroying osteoclasts and cells in the bone microenvironment cause the release of TGFbeta from the mineralized bone matrix which could act systemically on muscle to induce RyR1 remodeling.

Mice with PC-3 and LuCAP23.1 in castrate and hypogonadal states are studied for the following experiments to address the question of whether or not factors are released from bone that lead to muscle weakness:

1) extraskeletal tumors to bone metastases;

2) osteolytic bone metastases with osteoblastic;

3) cachexia with normal weight; and

4) bone metastases treated with bone resorption inhibitors.

To determine whether primary prostate cancer without bone metastases causes skeletal muscle weakness, mice are inoculated with human prostate cancer cells (primary tumor), and compared with non-tumor bearing mice (normal) and mice inoculated with the same prostate cancer cells via intracardiac route (bone metastases). Mice are assessed for muscle function, as disclosed herein, and cytokine measurements are made by multiplex. Human and mouse TGFbeta are measured in serum to determine the relative contribution of tumor and host TGFbeta production to the muscle phenotype.

To determine whether osteoblastic bone metastases are associated with muscle weakness, data obtained from the experiments described hereinabove are analyzed in which mice bearing osteolytic tumors are compared to mice bearing osteoblastic tumors.

To determine whether muscle weakness occurs in the absence of cachexia, data obtained from the experiments described hereinabove are analyzed in which mice bearing bone metastases with cachexia are compared with mice bearing bone metastases without cachexia. Further, a time course is performed in mice bearing bone metastases in order to analyze muscle function before the development of cachexia. In this experiment, mice are inoculated with tumors into the left cardiac ventricle on day 0 (when aged 4 weeks old). Seven mice are sacrificed each week to analyze muscle function as described herein. As mice do not start to lose weight until after 2 weeks post tumor inoculation (see FIGS. 3 and 4), muscle function and biochemistry from mice 1, 2, 3 and 4 weeks post tumor inoculation as well as in normal mice are analyzed and compared.

To determine whether inhibitors of bone resorption in vivo ameliorate skeletal muscle weakness in mice osteolytic prostate cancer bone metastases, mice are inoculated with prostate cancer via left cardiac ventricle and treated with the bisphosphonate zoledronic acid or osteoprotegerin to block osteoclastic bone resorption. Treatment begins at the time of tumor inoculation and continued throughout the experiment. Mice are assessed for bone metastases and muscle function as described herein.

Serum cytokines (by multiplex) and TGFbeta are measured. Since zoledronic acid and osteoprotogerin inhibit osteoclastic bone resorption by different mechanisms, it can be distinguished if the source of the TGFbeta and cytokine production is from the osteoclast.

Zoledronic acid inhibits bone resorption by inducing osteoclast apoptosis while osteoprotogerin does so by inhibiting RANKL-induced osteoclast formation. Thus, osteoclasts are still present and able to secrete factors, albeit unable to resorb bone, in the presence of zoledronic acid while osteoclasts are absent after treatment with osteoprotogerin. If the TGFbeta source is predominantly osteoclasts, it should be reduced in mice treated with osteoprotogerin compared with mice treated with zoledronic acid or control.

### RESULTS AND INTERPRETATIONS

The inventors have previously reported that oxidation of RyR1 (as occurs during normal aging) results in leaky RyR1 channels that cause impaired muscle force production and muscle weakness (Andersson et al., 2011). These effects are due to the oxidation induced loss of the stabilizing subunit calstabin 1 from the RyR1 channel complex. Since metastatic prostate cancer is also associated with a high level of oxidation. RyR1 is likely oxidized and leaky in prostate cancer models. The RyR1 mediated intracellular Ca²⁺ leak results in muscle damage and impaired force production as the inventors have previously shown in a number of systems including muscular dystrophy (Bellinger et al., 2009) and sarcopenia (Andersson et al., 2011).

Surprisingly, the inventors found that TGFbeta inhibitor SD-208 can improve prostate cancer related skeletal muscle weakness. Increased muscle specific force could induce bone fractures, which can be assessed using X-ray as described (Holstein et al., 2009; Paulus et al., 2001). To ensure that the effect of SD-208 is specific to TGFbeta, rather than other members of the TGFbeta superfamily such as myostatin or activin, the results of SD-208 treatment could be compared with a neutralizing antibody to TGFbeta from Genzyme, and is currently in clinical trials for fibrosis. Also, a skeletal muscle-specific deletion of TGF-3 receptor 2 (as a genetic test of the role of TGFbeta signaling in promoting oxidation of RyR1 and SERCA and prostate cancer associated muscle weakness), can be crossed into Rag−/− background and used to study prostate tumor models, RyR1 and SERCA biochemistry and function, and skeletal muscle function.

It should be understood that various changes and modifications to the methods and compositions described herein are possible without departing from the spirit and scope of the invention. Variations and modifications that can be made without departing from the spirit and scope of the invention will be apparent to those skilled in the art, and all such variations and modifications are within the scope of the invention.

What is claimed is:

1. A method of treating or preventing muscle weakness in a subject with cancer in need thereof, which comprises administering to the subject a therapeutically or prophylactically effective amount of an inhibitor of TGFbeta signaling.

2. The method of claim 1 wherein the effective amount of the TGFbeta signaling inhibitor either decreases the open probability of the RyR1 channel, decreases calcium leak through the RyR1 channel, decreases Ca²⁺ current through the RyR1 channel, increases the affinity with which calstabin 1 binds to RyR1, or decreases dissociation of calstabin 1 from RyR1.

3. The method of claim 1, wherein the TGFbeta signaling inhibitor is a TGFbeta antibody.

4. The method of claim 1, wherein the TGFbeta signaling inhibitor is selected from the group consisting of 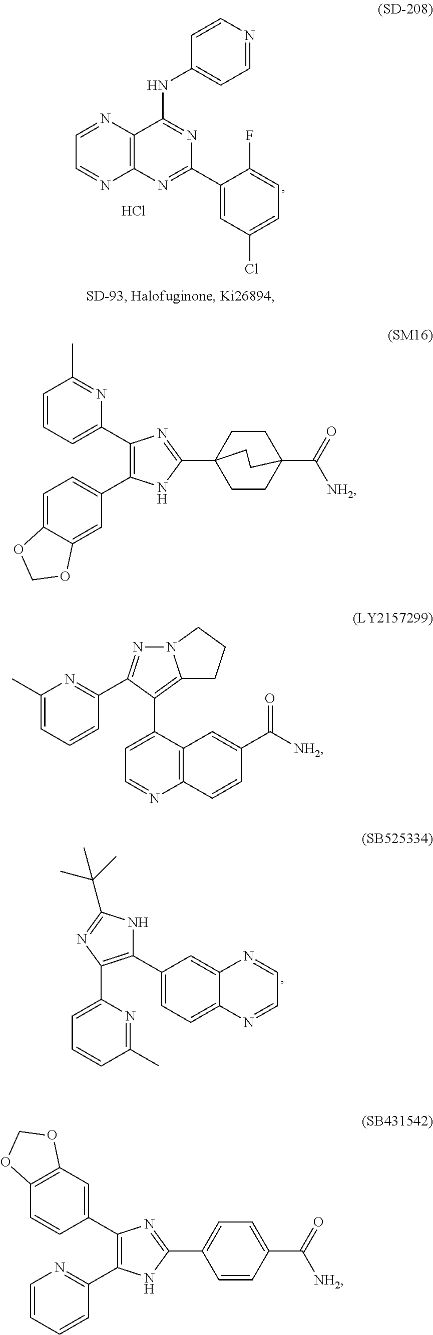 [SMILES: Fc1ccc(Cl)cc1-c1nc(Nc2ccncc2)c2nccnc2n1 ; Cl ; Cc1cccc(-c2nc(C34CCC(C(N)=O)(CC3)CC4)[nH]c2-c2ccc3c(c2)OCO3)n1 ; Cc1cccc(-c2nn3c(c2-c2ccnc4ccc(C(N)=O)cc24)CCC3)n1 ; Cc1cccc(-c2nc(C(C)(C)C)[nH]c2-c2ccc3nccnc3c2)n1 ; NC(=O)c1ccc(-c2nc(-c3ccc4c(c3)OCO4)c(-c3ccccn3)[nH]2)cc1] 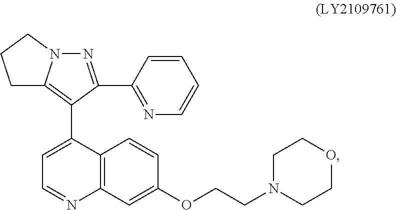 [SMILES: c1ccc(-c2nn3c(c2-c2ccnc4cc(OCCN5CCOCC5)ccc24)CCC3)nc1]
1D11, and AP 11014.

5. The method of claim 4, wherein the TGFbeta signaling inhibitor is SD-208.

6. The method of claim 1, wherein the cancer is selected from the group consisting of breast, prostate, pancreatic, lung, colon, and gastrointestinal cancers.

7. The method of claim 1, wherein the subject is a mammal selected from the group consisting of primates, rodents, ovine species, bovine species, porcine species, equine species, feline species and canine species.

8. The method of claim 1, wherein the subject is a human, the cancer is breast cancer, and the TGFbeta signaling inhibitor is SD-208 and is administered to the subject at a dose sufficient to restore or enhance binding of calstabin 1 to RyR1.

9. The method of claim 1, wherein the TGFbeta signaling inhibitor is administered by a route selected from the group consisting of parenteral, enteral, intravenous, intraarterial, intracardiac, intra intrapericardial, intraosseal, intracutaneous, subcutaneous, intradermal, subdermal, transdermal, intrathecal, intramuscular, intraperitoneal, intrasternal, parenchymatous, oral, sublingual, buccal, rectal, vaginal, inhalational, and intranasal.

10. The method of claim 9, wherein the TGFbeta signaling inhibitor is administered using a drug-releasing implant.

11. The method of claim 1, wherein the TGFbeta signaling inhibitor is SD-208 administered to the subject at a dose of from about 25 mg/kg/day to about 100 mg/kg/day.

12. The method of claim 1, wherein the TGFbeta signaling inhibitor is administered in a pharmaceutical composition or medicament that includes an excipient or carrier.
